(19) United States
(12) Patent Application Publication
(10) Pub. No.: US 2018/0078143 A1
(43) Pub. Date: Mar. 22, 2018

(54) PHOTO-ACOUSTIC IMAGING APPARATUS AND METHODS OF OPERATION

(71) Applicant: NANYANG TECHNOLOGICAL UNIVERSITY, Singapore (SG)

(72) Inventors: Manojit PRAMANIK, Singapore (SG); Paul Kumar UPPUTURI, Singapore (SG)

(73) Assignee: NANYANG TECHNOLOGICAL UNIVERSITY, Singapore (SG)

(21) Appl. No.: 15/561,018

(22) PCT Filed: Mar. 22, 2016
(86) PCT No.: PCT/SG2016/050130

§ 371 Date: Sep. 22, 2017

(30) Foreign Application Priority Data
Mar. 26, 2015 (SG) 10201502381X

(51) Int. Cl. A61B 5/00

(52) U.S. Cl. CPC A61B 5/0095; A61B 8/4483; A61B 5/0073; A61B 5/0062

(57) ABSTRACT

A photo-acoustic imaging apparatus (300) is provided for imaging a region (318) of a subject (306). The apparatus comprises: a light source (310) for directing light (314) at the region of the subject. A photo-acoustic transducer (320) senses photo-acoustic signals (404) induced in the region of the subject by the laser light, the photo-acoustic transducer being immersed in an ultrasound coupling medium and arranged to scan the region of the subject and to move in a curvilinear path (402) around the region of the subject. The light source is disposed within a volume (346) defined, at least in part, by the curvilinear path.

The present application claims priority from Singapore Patent Application No. 10201502381X, the contents of which are hereby incorporated by reference in their entirety.

The invention relates to a photo-acoustic imaging apparatus for imaging a region of a subject. The invention also relates to a method of imaging a region of the subject. The invention has particular, but not exclusive, application in a low-cost, high-speed, portable pulsed laser diode photo acoustic tomography system.

Photo-acoustic tomography (PAT) is a promising non-ionizing hybrid imaging modality combining high optical contrast and ultrasonic resolution for various clinical applications, such as, breast imaging, brain imaging, molecular imaging, vasculature imaging in small animals etc. [1-8]. In photo-acoustic tomography a short laser pulse irradiates the tissue. Due to absorption of incident energy by the tissue chromophores (such as melanin, red blood cells, water etc.), there is a local temperature rise, which in turn produces pressure waves emitted in the form of acoustic waves. A wideband ultrasound transducer receives the photo-acoustic signals outside the tissue boundary. The photo-acoustic waves are acquired at various positions around the tissue boundary. Generally, a circular scanning geometry in orthogonal excitation mode is preferred for deep tissue imaging. Reconstruction techniques [9-12] are used to map the initial pressure rise within the tissue from the measured photo-acoustic signals.

In photo-acoustic tomography systems, Nd:YAG lasers have been widely used as excitation sources which can provide 5-10 ns pulses with pulse energy of tens of milliJoules. This laser pumps a second stage optical parametric oscillator (OPO) or dye laser to produce pulses in the near infrared (NIR) region. Since in the NIR window the optical absorption is weak, it has been widely used for deep-tissue imaging. However, these (Nd:YAG/OPO or dye-based) lasers are expensive, bulky, and are not suitable for high-speed imaging with, say, a single-detector due to the low-repetition rate (^{˜}10 Hz for ^{˜}100 mJ per pulse energy) [7, 13-15]. The pump laser and the OPO/dye lasers needs to be precisely aligned for optimum production of laser output, therefore, such systems also need to be housed on vibration-isolation optical tables during operation. So, clinical translation of such systems is challenging. Although, recently a few companies have developed portable OPO lasers, suitable for photo-acoustic tomography, but are even more expensive. The pulse repetition rate is still a bottleneck for high speed PAT imaging, as they can only operate at ^{˜}10 Hz (^{˜}100 mJ per pulse). It is possible to use higher repetition rate lasers (20-50 Hz), but then there is a need to sacrifice the laser energy output. To achieve high-speed imaging, several scanning geometries were demonstrated. In a circular scanning geometry, photo-acoustic signals are collected while the single-element photo-acoustic transducer, for example an ultrasonic transducer (UST), rotates around the sample in full circle. Since one detector rotates around the sample the imaging speed is rather slow. Therefore, photo-acoustic tomography systems based on linear [16-18], semi-circular [19, 20], circular array [21, 22] of ultrasonic transducers have been used for high speed imaging. They do not require scanning and hence can improve imaging speed (still limited to a maximum imaging speed of 10 frames per second as the laser is operating at that frequency). However, such array transducers are very expensive and not readily available. Typical an ultrasound array will have 128/256 elements. To acquire data from all the channels simultaneously one requires 128/256 channels signal acquisition system (digitization and amplification) with a high sampling rate (>20 Ms/s). Such electronics are also very expensive.

In recent years, photoacoustic imaging was successfully demonstrated using high-repetition rate pulsed laser diode (PLD) as the excitation source. A fibre based illumination with cylindrical scanning geometry was demonstrated on phantoms using pulsed near infrared laser diode based photo-acoustic tomography [23, 24]. In vivo photo-acoustic imaging of superficial human blood vessels at a depth of ^{˜}1 mm below the skin was achieved [25]. Optical resolution-photoacoustic microscopy (OR-PAM) with a pulsed laser diode was reported [26, 27]. Photo-acoustic imaging was demonstrated by intensity-modulation of continuous wave output from 785 nm laser diode [28, 29]. Recently in-vivo images at frame rates of 10 fps were obtained using an 805 nm laser diode with 0.5 mJ pulse energy. The in vivo imaging depth was about 4-7 mm while operating at the maximum permissible exposure (MPE) of 1.5 mJ/cm² for a PRF of 210 Hz. An imaging depth of up to 15 mm was demonstrated in phantoms for a frame rate of 0.43 Hz [30]. Until now, the PAT systems reported are either bulky, expensive, low-speed or low-penetration which makes them not ideal for translating into clinical use.

A known technique from [14] is illustrated in FIG. 1. A Nd:YAG laser is implemented in a circular scanning technique for small-animal imaging.

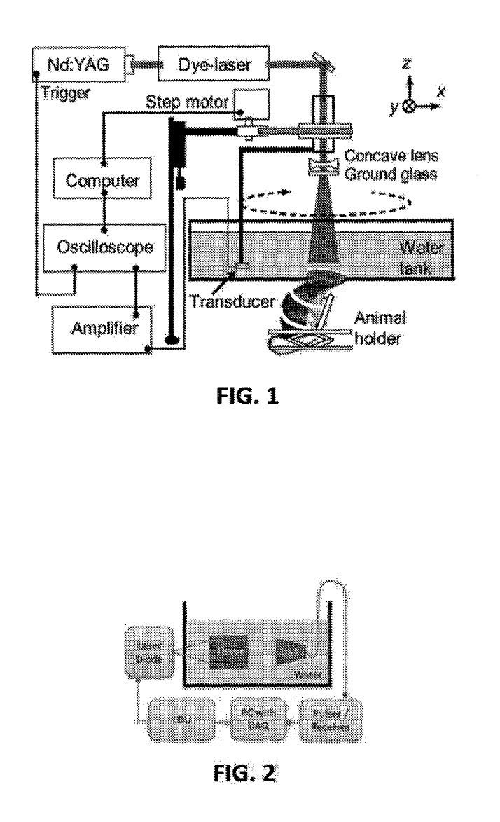

Another known technique is illustrated in FIG. 2. A laser diode, driven by a laser driver unit, irradiates a tissue sample immersed in water. An ultrasonic transducer senses photo-acoustic signals, which are transmitted to a pulser/receiver. The received signals are processed and passed to a computing device having a data acquisition unit.

United States Patent Publication No. US 2008/0173093 A1 illustrates a system and method for photoacoustic tomography of a sample, such as a mammalian joint, includes a light source configured to deliver light to the sample, an ultrasonic transducer disposed adjacent to the sample for receiving photoacoustic signals generated due to optical absorption of the light by the sample, a motor operably connected to at least one of the sample and the ultrasonic transducer for varying a position of the sample and the ultrasonic transducer with respect to one another along a scanning path, and a control system in communication with the light source, the ultrasonic transducer, and the motor for reconstructing photoacoustic images of the sample from the received photoacoustic signals.

The invention is defined in the independent claims. Some optional features of the invention are defined in the dependent claims.

Implementation of the techniques disclosed herein may provide significant technical benefits. For instance, as disclosed herein, there is a photo-acoustic apparatus that may be compact, affordable, having high-speed and deeper imaging capabilities in biological tissues which could make the photo-acoustic tomography system a standard tool for clinical applications. In one exemplary system, there is a pulsed laser diode photo-acoustic tomography system that may integrate a low-cost, compact pulsed laser diode source with a single-detector circular scanner. The exemplary system(s) is/are demonstrated for high-speed and deep-tissue imaging on blood embedded in biological tissues. The resolution, imaging-speed, imaging-depth and image-quality of the pulsed laser diode photo-acoustic tomography system with a traditional Nd:YAG/OPO based PAT (OPO-PAT) system is later compared.

It is possible to implement the technique(s) described herein using a single photo-acoustic detector in a curvilinear (for example, circular) scan geometry to form photo-acoustic images, a relatively inexpensive option. To improve the imaging speed, it is possible to use a high repetition rate laser.

Further advantages from implementation of the techniques disclosed herein include the following.

In existing photo-acoustic tomography systems the laser light enters the scanner from outside through free space optics or fibre bundle. In the techniques disclosed herein, the pulsed diode laser source itself is integrated inside the circular scanning geometry. So, it is not necessary to bring light from outside the envelope/volume of the scanning geometry to the sample. Thus, the techniques disclosed herein can be considered to provide a system in which a light source, such as a pulsed laser, is integrated inside the scanning geometry.

The existing photo-acoustic tomography systems combine low repetition rate (pulsed Nd:YAG) laser and single detector circular scanning which may be considered to make the data acquisition time quite long. For example, [14] presents a circular scanning PAT system which takes around 24 min to form a single 2-D slice. With the techniques described herein, it is possible to combine a high repetition rate diode laser and fast scanning single detector in a curvilinear such as circular geometry to achieve high-speed imaging. The disclosed system(s) can provide an imaging time of three seconds to form a 2-D image. No existing single detector photo-acoustic tomography system has demonstrated such high-speed imaging so far.

The existing photo-acoustic tomography systems use lasers which are expensive (typically costing more than USD 100 K), bulky (typically being heavier than 100 kg weight) and non-portable. If using the techniques disclosed herein, it is possible to realise a low-cost (projected at approximately USD 15K), and light-weight (approximately 200 gm) laser diode to make the overall system compact, portable, and less-expensive for, for example, pre-clinical applications.

In [14], this uses a bulky, expensive, and low-repetition rate laser as excitation source out-side the scanner. The techniques disclosed herein may implement a compact, low-cost, and high repetition-rate laser “integrated” inside the scanner itself. No such photo-acoustic system that integrates a laser such as a laser diode inside a single detector circular scanning geometry has been reported so far.

As shown in FIG. 2, pulsed laser diodes have already been used in photo-acoustic tomography, but such systems have not and cannot work using a single element transducer with curvilinear (e.g. circular) scanning geometry. Moreover, these reported laser diodes can generate pulses with low-energy (^{˜}0.5 mJ) and hence they could achieve very low imaging depth maximum ^{˜}4 mm. In one arrangement as described, it is possible to generate pulses with energy (^{˜}1.45 mJ) and hence achieve imaging depth ^{˜}4 cm which is good enough for small animal pre-clinical study.

United States Patent Publication No. US 2008/0173093 A1 emphasises a photo-acoustic tomography system that uses an array of transducers to decrease data acquisition time by avoiding scanning. But such transducer arrays are expensive and not readily available in the market. The techniques disclosed herein may use a cost-effective single-element transducer configured to scan at high speed in a circular geometry. 3D imaging can be achieved either by moving transducer or sample vertically in a controlled manner.

One of the techniques described below allows variation in the distance between the pulsed laser and the subject/sample. This can be a potentially significant benefit in that the laser can be used to illuminate (irradiate) only the area of interest especially when studying different sized samples so that the laser light energy will not be wasted. In existing photo-acoustic tomography systems, the changing of the illumination area is achieved by using an additional optical lens system.

One of the techniques described below allows for the photo-acoustic transducer to be mounted using, for example, square shaped rods, in such a way that once the transducer is mounted it faces the centre of the scanning area. This may be configured so that the transducer faces the centre of the scanning area at all times, (when, for example, continuous scanning is performed) or at discrete points along the scanning path of travel. This overcomes the tedious problem of aligning the transducer, a problem which is particularly acute when the transducer and/or the sample/subject are immersed in a liquid such as water. It is beneficial for the transducer to point towards the scanning area, particularly the centre of the area in order to accurately reconstruct the photo-acoustic tomography images. Using this mounting arrangement, no further alignment of the transducer is required after the initial setup. Therefore, the transducer can be considered to be automatically aligned.

Overall, implementation of the techniques described herein may provide for the following novel features: 1) an “integrated” pulsed diode laser inside the scanner; 2) a “portable” PAT system; 3) Faster imaging (e.g. three seconds) speed using a single element transducer; 4) a centre aligned transducer holder; 5) smaller scanning radius leading to a smaller water tank, optionally with the use of ultrasound reflector, as overall this reduces the footprint of the scanner.

The invention will now be described, by way of example only, and with reference to the accompanying figures in which:

FIG. 1 is a schematic block diagram illustrating the architecture of a known photo-acoustic tomography system;

FIG. 2 is a schematic block diagram illustrating the architecture of a second known photo-acoustic tomography system;

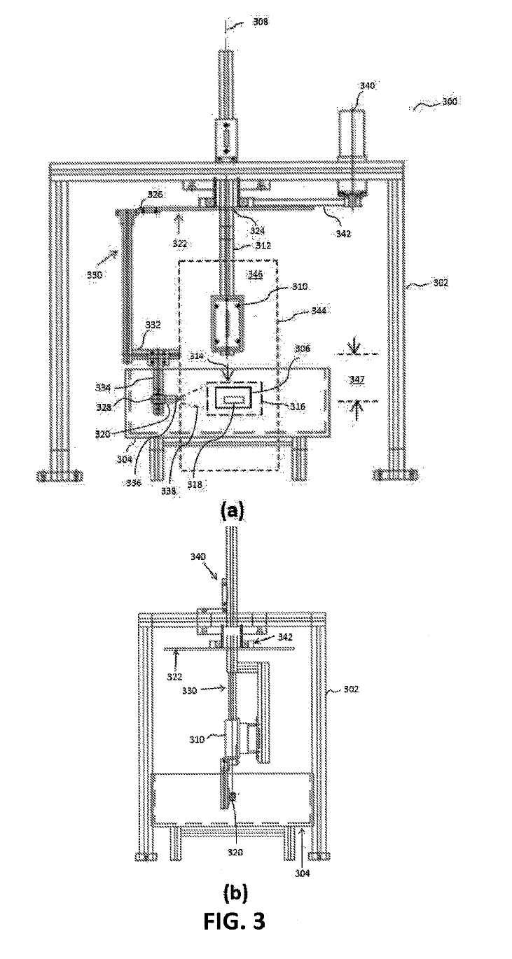

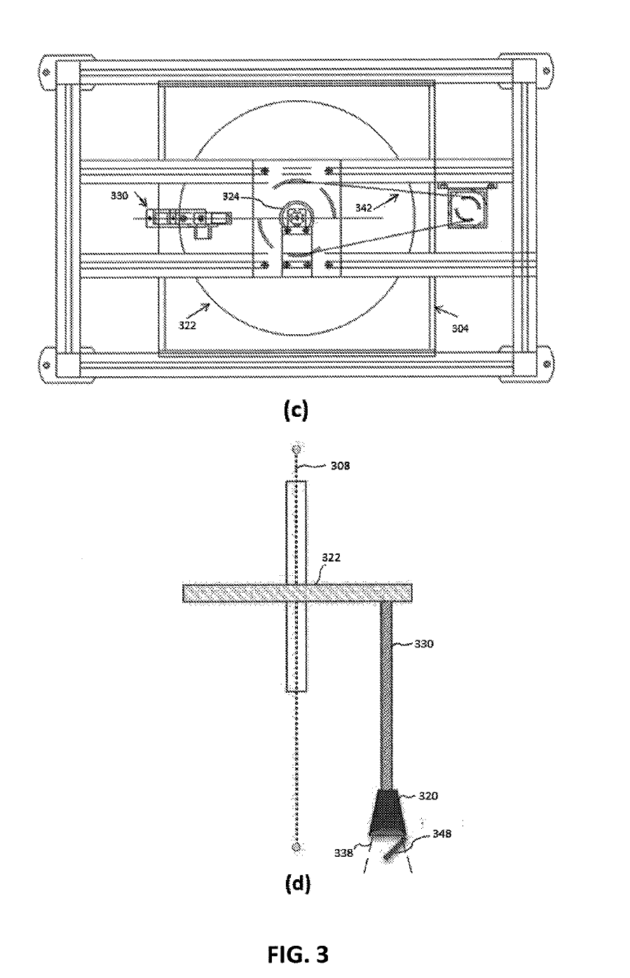

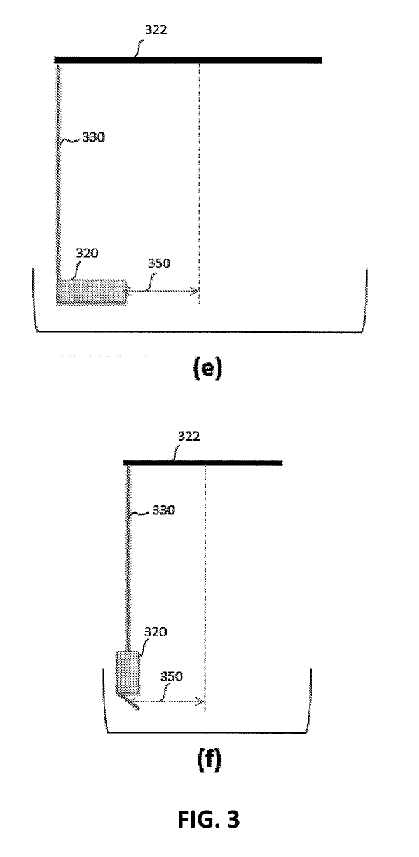

FIGS. 3(a), (b) and (c) provide a series of views of, respectively, a front view, a side view and a plan view of a novel photo-acoustic imaging apparatus, and FIGS. 3(d), (e) and (f) provide a series of views of an alternative arrangement for the photo-acoustic sensor and an advantage of this arrangement;

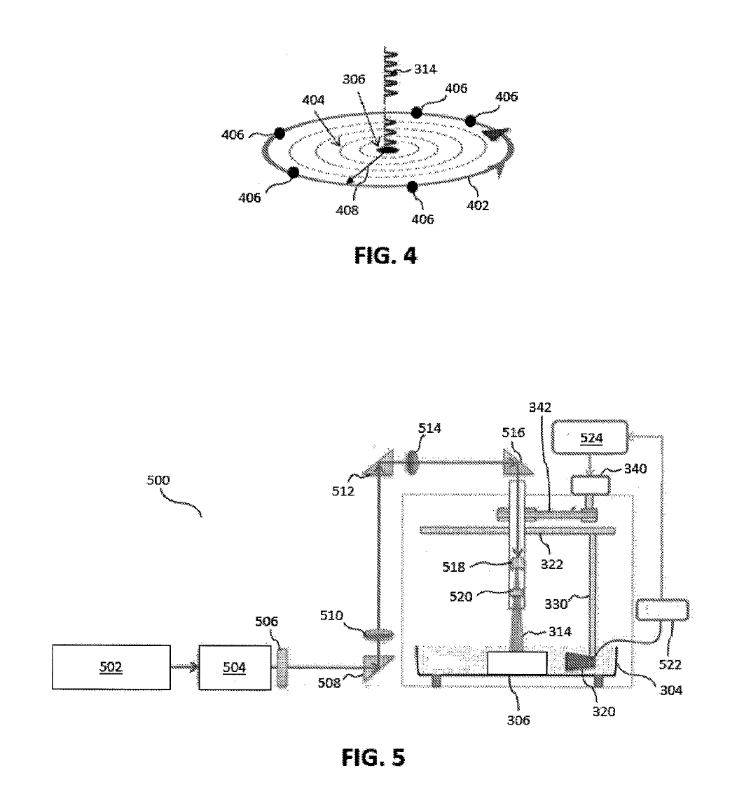

FIG. 4 is a schematic diagram illustrating the generation of photo-acoustic signals;

FIG. 5 is a schematic diagram illustrating an experimental setup for verifying the results obtained by the apparatus of FIG. 3;

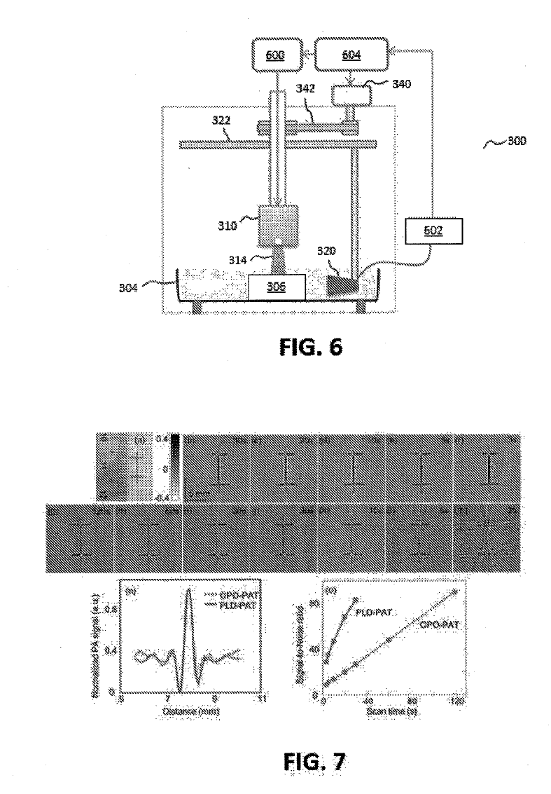

FIG. 6 is a schematic diagram illustrating a novel photo-acoustic imaging apparatus;

FIG. 7 illustrates a series of reconstructed images obtained implementing the techniques disclosed herein, the scan time and the line scan profiles;

FIG. 8 illustrates a series of reconstructed images obtained implementing the techniques disclosed herein, the line scan profile and the signal-to-noise ratio;

FIG. 9 illustrates seriously claim Melissa the a series of reconstructed images obtained implementing the techniques disclosed herein, and the stack of tissue layers inside which the sample/subject was disposed;

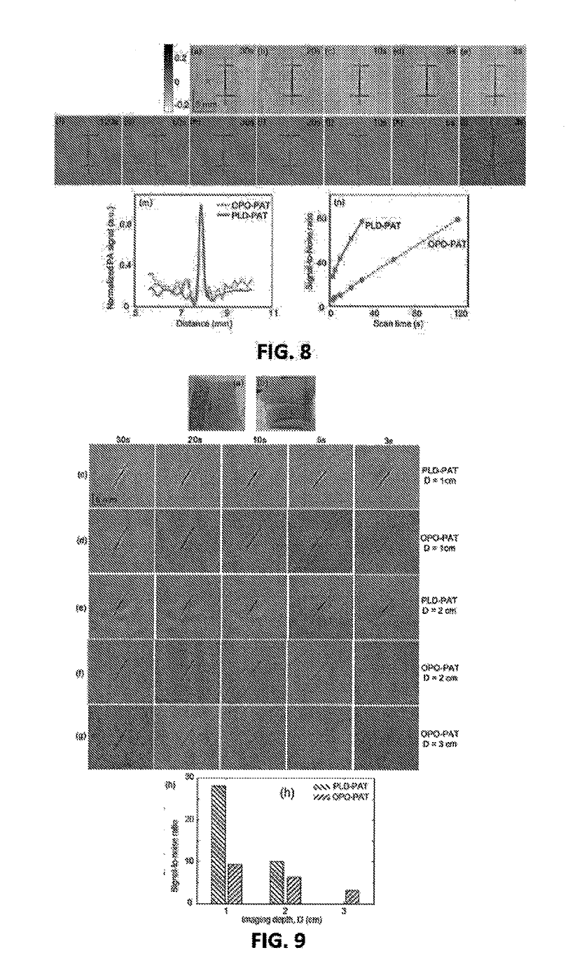

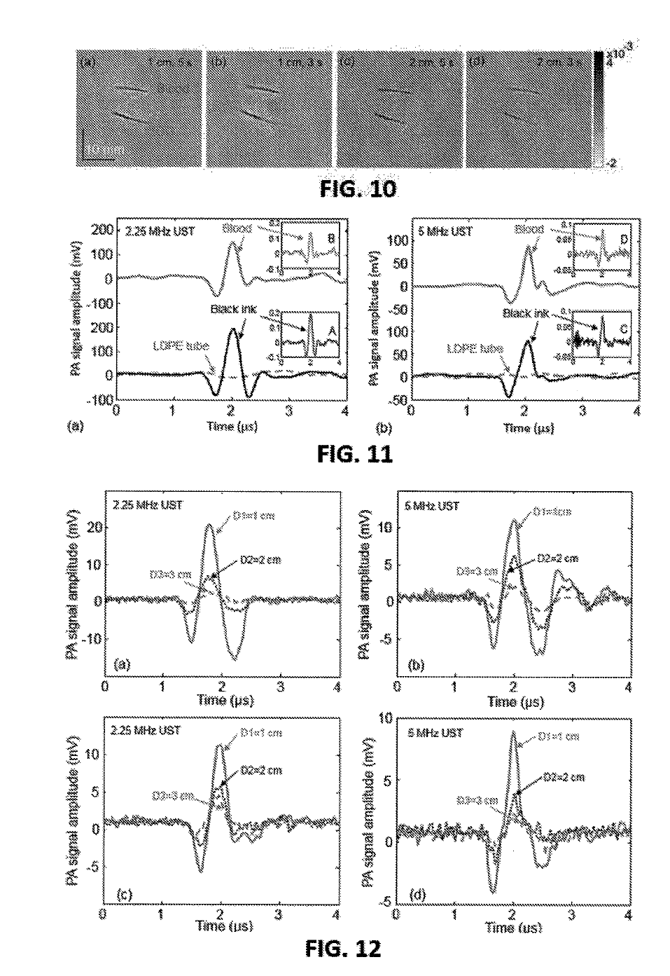

FIG. 10 illustrates a series of reconstructed images obtained implementing the techniques disclosed herein;

FIG. 11 illustrates a series of optoacoustic signals acquired implementing the techniques disclosed herein;

FIG. 12 illustrates a series of optoacoustic signals acquired implementing the techniques disclosed herein;

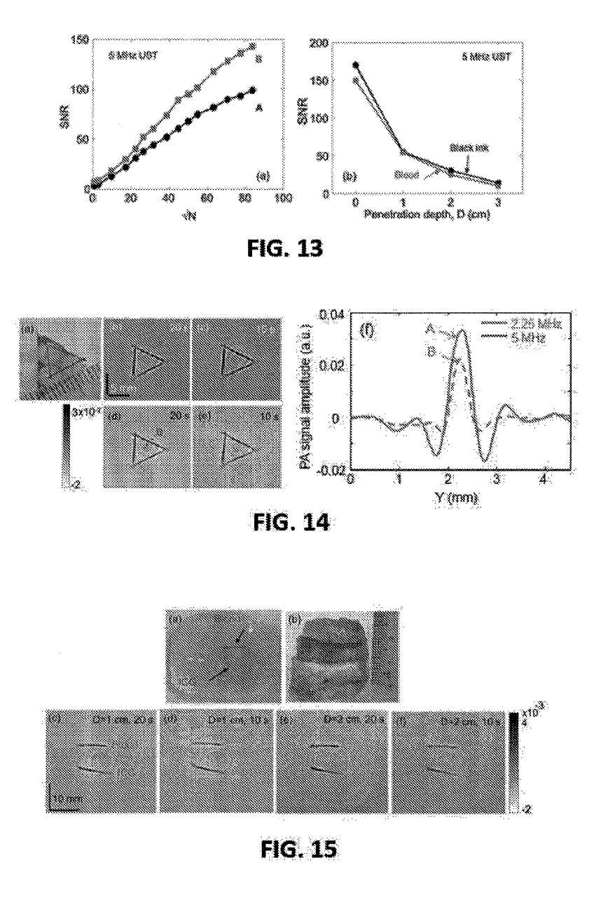

FIG. 13 illustrates the signal-to-noise ratio of optoacoustic signals acquired implementing the techniques disclosed herein;

FIG. 14 illustrates a series of reconstructed images obtained implementing the techniques disclosed herein;

FIG. 15 illustrates a series of deep tissue images obtained implementing the techniques disclosed herein;

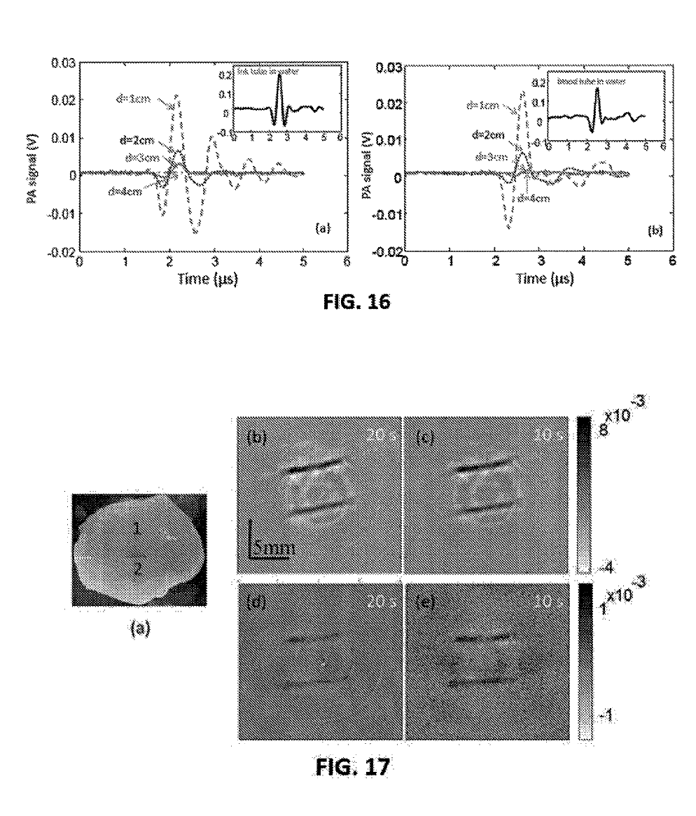

FIG. 16 illustrates a series of photo-acoustic signals obtained implementing the techniques disclosed herein;

FIG. 17 illustrates a photo of tissue phantom used in an instance of implementing the techniques disclosed herein, and a series of reconstructed images therefrom;

FIG. 18 illustrates a series of reconstructed images obtained implementing the techniques disclosed herein;

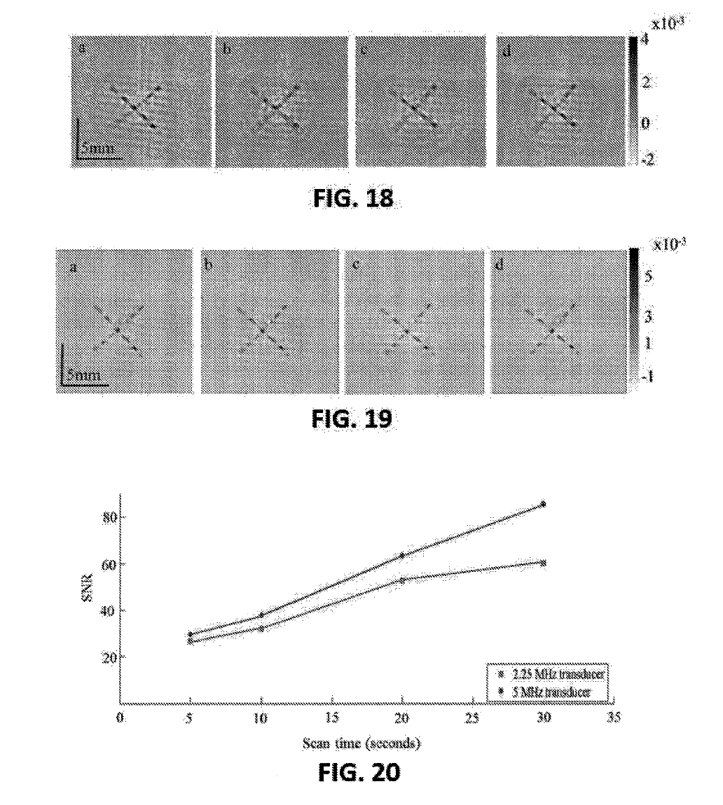

FIG. 19 illustrates a series of reconstructed images obtained implementing the techniques disclosed herein;

FIG. 20 illustrates the signal to noise ratio of signals obtained implementing the techniques disclosed herein across a range of scanning speeds;

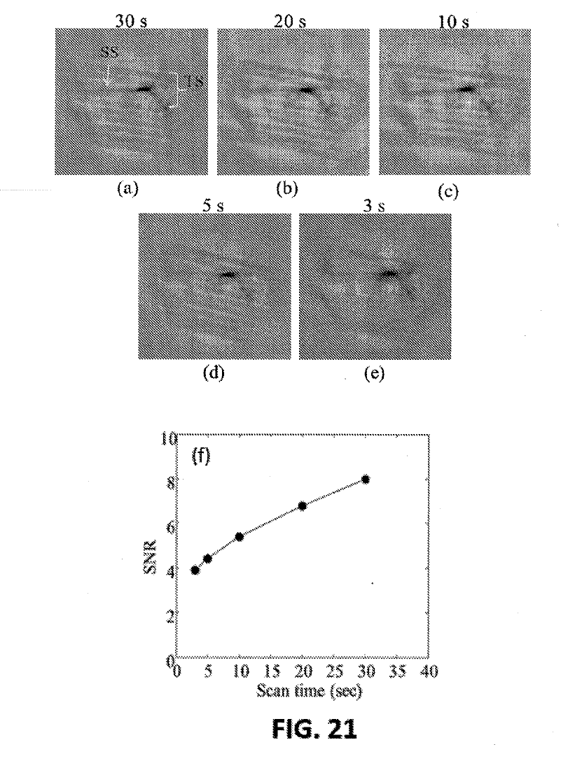

FIG. 21 illustrates a series of reconstructed images obtained implementing the techniques disclosed herein and signal-to-noise ratio as a function of scan time;

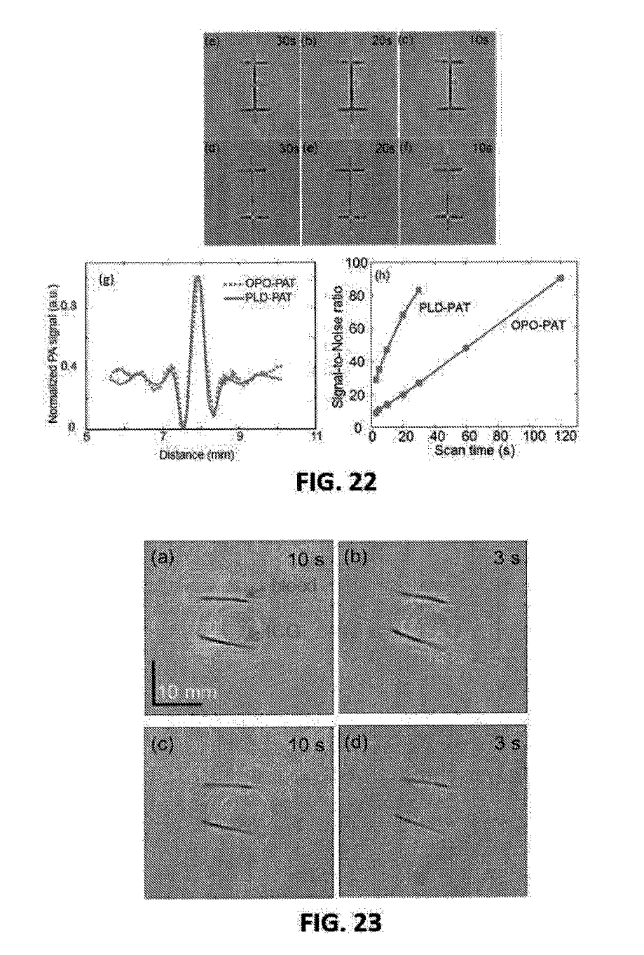

FIG. 22 illustrates a series of reconstructed images obtained implementing the techniques disclosed herein and corresponding signal to noise ratio plots; and

FIG. 23 illustrates a series of reconstructed images obtained implementing the techniques disclosed herein.

Turning now to FIG. 3, an exemplary photo-acoustic imaging apparatus 300 implementing the techniques disclosed herein is illustrated. FIG. 3 provides a series of views of apparatus 300: FIG. 3(a) provides a “front” elevational view; FIG. 3(b) provides a “side” elevational view; and FIG. 3(c) provides a top or plan view.

Taking FIG. 3(a) first, photo-acoustic imaging apparatus 300 comprises a support frame 302 for supporting a number of individual components of the exemplary apparatus 300. Subject holder 304 may be provided to hold/support/contain a subject 306 (e.g. a sample) to be imaged (or at least a region of the subject is to be imaged).

In this example, photo-acoustic imaging apparatus 300 has a central axis 308, the importance of which will become apparent. Apparatus 300 further comprises a light source 310, such as a pulsed laser diode, which may be mounted in the central axis 308, using mount 312, as described in more detail below.

Pulsed laser 310 emits laser light (pulses) 314 to irradiate/illuminate scan area 316. As is apparent from FIG. 3(a), subject 306 is disposed within the scan area 316. Thus, when subject 306 is disposed in this scan area 316, it will be irradiated/illuminated by the laser light 314 with a view to imaging at least region 318 of the subject 306.

Photo-acoustic signals induced in subject 306 (or at least region 318 thereof) when illuminated by laser light 314 are picked up by photo-acoustic sensor 320 immersed in an ultrasound coupling medium (omitted from the figure for the sake of clarity) such as water, mineral oil or a gel, such as an ultrasound gel. A scanning plate 322 has a central hole 324 therethrough, and is aligned through hole 324 on central axis 308 of apparatus 300. Scanning plate 322 provides a mounting point 326 at or on which a holder 328 for the photo-acoustic transducer 320 may be fixed/mounted. This photo-acoustic transducer holder comprises a first member 330, which extends “downwards” (in the direction towards the subject holder 304 and the base of the support frame 302). In at least one arrangement, this first member 330 extends downwards in or near the vertical plane. The photo-acoustic transducer holder comprises a second member 332, extending from first member 330. In at least one arrangement, this second member 332 extends from first member 330 in or near the horizontal plane. The photo-acoustic transducer holder comprises a third member 334, extending from second member 332. In at least one arrangement, this is third member 334 extends from second member 332 in or near the vertical axis. Thus, it may be considered a second vertical member 332.

It is to be noted that, in this example, the subject 306 is also immersed in/disposed within the ultrasound coupling medium, but this is not required for system operation.

Photo-acoustic transducer 320, as may be expected, comprises a sensing component 336, such as a sensor array, which performs the detection of the photo-acoustic signals. Sensor 336 may be considered to have a sensing “field of view” 338, a field in which or from which the sensor is capable of picking up photo-acoustic signals generated therein, and propagating towards the sensor.

Scanning plate 322 is arranged to rotate about central axis 308. Apparatus 300 comprises a driving motor 340 for providing driving power to a mechanical linkage, such as a pulley system, 342 which transmits motive power from the motor to rotate the scanning plate 322.

Scanning plate 322, when rotating, defines a curvilinear path, such as a circular path. As will be apparent from FIG. 3, this exemplary scanning plate is circular, but other arrangements, such as elliptical, semi-circular and other scanning plate geometries are contemplated.

When the scanning plate 322 is driven to rotate, photo-acoustic transducer 320 also rotates, given the fact this is mounted to scanning plate 322. Thus, the photo-acoustic transducer is moved in a curvilinear path which, in this example, is a circular path 402, as seen in FIG. 4. Thus, the photo-acoustic transducer 320 is arranged to scan around the subject 306 and it may scan around the sample in a plane perpendicular to the emitted laser light. As laser light (pulses) 314 irradiates subject 306, photo-acoustic signals 404, photo-acoustic waves or signals (which may be referred to as A-lines) are generated, and detected by photo-acoustic transducer 320 as it traverses the curvilinear path 402. In instances where these received photo-acoustic signals 404 are averaged, the averaged values of these signals may also be called “A-lines” and/or “averaged A-lines”.

In this respect, at least several exemplary modes of operation for the scanning are contemplated. In a first arrangement, scanning plate 322 is driven by motor 340 to rotate continuously, with photo-acoustic sensor 320 scanning for photo-acoustic signals 404 continuously. In a second arrangement, scanning plate 322 is, again, driven continuously by motor 340, but photo-acoustic sensor 320 scans for photo-acoustic signals 404 at discrete points 406 along the curvilinear path 402. In a third arrangement, scanning plate 322 is driven in steps, moving from scanning point 406 to scanning point 406, with the scanning plate pausing at least momentarily while photo-acoustic sensor 320 is at each scanning point 406 to detect photo-acoustic waves 404.

Referring again to FIG. 3(a), dashed lines 344 define a volume 346 within which the pulsed laser 310 is disposed. In the example given, the vertical dashed lines 344 are incident on the curvilinear path 402. That is, it is the curvilinear path of the photo-acoustic transducer 302 which defines the vertical lines. Significantly, pulsed laser 310 is positioned within these vertical lines, within volume 346 thus providing a truly “integrated” photo-acoustic sensing apparatus thus obviating the requirement for the provision of a complicated light circuit for conveying laser light from outside the envelope of the volume to the subject 306. As mentioned above, this arrangement may provide for a portable, lightweight and reduced-cost photo-acoustic sensing apparatus.

Therefore it will be appreciated that FIG. 3 in conjunction with FIG. 4 illustrates a photo-acoustic imaging apparatus 300 for imaging a region 318 of a subject 306, the photo-acoustic apparatus 300 comprising: a light source 310 for directing light 314 at the region 318 of the subject 306; and a photo-acoustic transducer 320 for sensing photo-acoustic signals 404 induced in the region 318 of the subject 306 by the light 314, the photo-acoustic transducer 320 being immersed in an ultrasound coupling medium (water, in this example) and arranged to scan the region 318 of the subject 306 and to move in a curvilinear path 402 around the region 318 of the subject 306; wherein the light source 310 is disposed within a volume 346 defined, at least in part, by the curvilinear path 402.

In the example given, the light source is a pulsed laser, but other arrangements are also contemplated. For instance, it is contemplated that photoacoustic techniques may be implemented using LEDs, especially as the technology of LEDs continues to improve [100].

A corresponding method is also described. That is, there is provided a method of imaging a region 318 of a subject 306, the method comprising: directing light 314 from a light source 310 at the region 318 of the subject 306; sensing photo-acoustic signals 404 induced in the region 318 of the subject 306 by the light 314 using a photo-acoustic transducer 320 immersed in an ultrasound coupling medium (e.g. water), the photo-acoustic transducer 320 scanning the region 318 of the subject 306 and moving in a curvilinear path 402 around the region 318 of the subject 306, wherein the light source 310 is disposed within a volume 346 defined, at least in part, by the curvilinear path 402.

In a useful setup, the apparatus 300 may comprise one or more of the following. Pulsed laser 310 is a pulsed laser diode, such as one provided by Quantel of France, which provides pulses of approximately 136 ns pulse width. These pulses may be provided in the near infrared part of the spectrum, with one particularly useful wavelength (or range of wavelengths) having been found by the inventors to be at or around 803 nm. The pulse energy may be of the order of 1.5 mJ, with a maximum 7 kHz repetition rate. Thus, the light source 310 comprises a high-repetition rate pulsed laser diode.

The mounting of the pulsed laser diode 310 may be such that it generates a rectangular beam which diverges with an angle of approximately 11.5°, and approximately 0.65° along the slow and fast axes respectively. Other shapes of laser beam may also be used, such as a circular shape. The pulsed laser diode 310 may be controlled by a laser driver unit which consists of a temperature controller such as the MTTC1410 model provided by LaridTech, a power supply, such as the Voltcraft 12 V power supply model PPS-11810, a variable power supply (to change the laser output power), and a function generator (to control the laser repetition rate). The pulse energy (0.2-1.4 mJ) and repetition rate (up to 7 kHz) can be controlled independently with a variable power supply (such as that provided by BASETech, model BT-153), and a function generator (such as FG250D, Funktionsgenerator), respectively. The function generator may provide a TTL (Transistor-Transistor Logic) signal to synchronize the data acquisition (DAQ) with the laser excitation.

In at least one arrangement, photo-acoustic transducer 320 is an ultrasonic transducer. Apparatus 300 may be arranged such that the subject holder 304 is filled with water so that the ultrasonic transducer and/or the subject are immersed therein for improved coupling of the photo-acoustic signals. It is possible to use two non-focus transducers (V323-SU/2.25 MHz, V309-SU/5 MHz, Olympus NDT) with 13 mm active area and ^{˜}70% nominal bandwidth, although other types and numbers of transducers may also be used. One particularly useful type of drive motor 340 is a step motor (such as that by Lin Engineering, Silverpak 23C). The arrangement of the holder for the ultrasonic transducer, particularly the arrangement of the members 330, 332, 334 as described above, may be particularly useful in that it can be used to align automatically the ultrasonic transducer in such a way that its sensor 336 is always facing the scan area 316, preferably the centre of the scan area. This may be particularly beneficial as it is a time-consuming process to align this manually, and if the photo-acoustic sensor 320 is not facing the scan centre it may result in inaccurate image reconstruction. This “automatic alignment” can be realised at all points along the curvilinear path (for example, for the continuous scanning technique described) or at least at the discrete points 406.

Thus, it will be appreciated that photo-acoustic imaging apparatus 300 is arranged for a sensor 336 of the photo-acoustic transducer 320 to face the region 318 of the subject 306 at plural points (i.e. the points 406 or at all points) along the curvilinear path 402.

The mechanical linkage 342 may comprise, as appropriate, a gear box, pulley or pulleys and belts to translate the (stepper) motor motion into the motion of the photo-acoustic sensor, in a curvilinear path such as a circular path. The photo-acoustic signals are subsequently amplified, and band pass filtered, as described above with reference to FIG. 6, by ultrasound signal receiver 602 unit (such as that of Olympus-NDT, 5072PR), and then digitised and recorded by the PC with data acquisition (DAQ) card 604 (such as that of GaGe, compuscope 4227). Usually, low-frequency ultrasound detectors (1-5 MHz) are used in photo-acoustic tomography, so the DAQ card may be operated at a sampling frequency of 25 Ms/s.

In a photo-acoustic system that uses single-element detector scanning, the photo-acoustic signal (A-line) 404 acquisition can be performed in a number of ways, as described above. In the above-described “stop-and-go” scanning method [31, 32], the motor moves the photo-acoustic sensor to one of pre-defined positions 406, collecting at least one and preferably multiple photo-acoustic signals, for the PC/DAQ to average them if necessary recording/saving the A-line(s), and moving to the next position 406. Additionally or alternatively, the photo-acoustic apparatus 300 is configured to perform a continuous scanning method [33]. In this, the motor rotates the photo-acoustic transducer continuously at predefined speed, collecting multiple photo-acoustic signals as the transducer is moving along the curvilinear path 402. The A-lines data may be transferred to the PC on the fly, or once the rotation is complete. Signal averaging can also be done later if needed. The inventors have found that the continuous scanning method is preferable in both the arrangements of FIG. 3 for the pulsed laser arrangement and FIG. 5 for the OPO-PAT method. Continuous scan is a faster operation, requiring less time to complete than the stop-and-go data acquisition method. It has been found to be possible that in the setup of FIG. 3, just three seconds is required for a full 360 degree rotation.

As will be described below, it is possible to obtain a photo-acoustic tomography scan in as short as three seconds using a continuous scanning mode of operation without any sacrifice in the image quality using the pulsed laser arrangement of FIG. 3. The image blur introduced due to continuous scanning has been found to be negligible compared to the imaging resolution or the subject being imaged. For example, for an object size of approximately 15 mm diameter, it will take around 10 μs (speed of sound=1.5 mm/μs) to record all the photo-acoustic signals 404 originating from the object in a single A-line. During this time, the maximal displacement of points within the object with respect to the photo-acoustic sensor surface can reach approximately 0.15 μm (for a 3 second scan time). This blur due to continuous scanning is negligible compared to the ultrasonic resolution of the system. With the arrangement of FIG. 3 it is possible to perform signal averaging before the reconstruction to reduce the computation load. For example, the total number of A-lines were reduced to 500 by signal averaging 42 signals (in case of 3 s scanning, the total number of A-lines=3×7000=21000; after averaging the A-lines=21000/42=500). This averaging may also introduce blur in the reconstructed image. However, the blur due to averaging is ^{˜}90 μm, which is also negligible compared to the resolution of the photo-acoustic tomography system as well as the size of the objects being imaged.

In another example, the object size is ^{˜}10 mm (diameter), it takes ^{˜}6.7 μs (speed of sound=1.5 mm/μs) to record a single A-line. During this time, the maximal displacement of points within the object with respect to the photo-acoustic sensor surface can reach ^{˜}0.04 μm (for a 10 second scan time). The signals were averaged before the image reconstruction to reduce the computation load. For example, total number of A-lines were reduced to 500 by signal averaging 140 signals (in case of 10 s scanning, total A-lines=10×7000=70000; after averaging A-lines=70000/140=500).

In OPO techniques, the A-line data during image reconstruction is not averaged as there are fewer A-lines when compared with the pulsed laser diode system, typically OPO runs at 10 Hz versus 7 kHz for the pulsed laser diode used here. Referring again to the discussion of FIG. 4, the A-lines data may be used to reconstruct the cross-sectional photo-acoustic image of the sample, and this may be done using a simple delay-and-sum back projection reconstruction algorithm. The transducer rotation, data collection, and the image reconstruction may be performed using MATLAB programs.

In terms of the averaging of the A-lines, the stepper motor rotates the photo-acoustic transducer continuously at a predefined speed, collecting multiple photo-acoustic signals as the transducer is moving. The A-lines may be saved once the rotation is complete, after which signal averaging can be done over a constant number of A-lines (such as consecutive A-lines) by the PC/DAQ. This may help to reduce the computation load during image reconstruction. For ‘t’ sec scanning and ‘P_{R}’ repetition rate, the number of A-lines after averaging, N=(P_{R}*t)/n, where n—is the number of A-lines averaged. For 3 sec scan time, 7000 Hz repetition rate, n=42 A-line averaging, the number of averaged A-lines would be N=500. For example instead of choosing n=42 if we choose n=21, then N will be 1000. So if n decreases, N increases. As N increases the reconstruction load also increases. On the flip side, if N becomes too low the reconstruction image quality suffers. So these parameters are selected as an optimisation between image quality and image reconstruction load. It has been found from the inventors' experience and from literature that an N value of between 400-1200 is optimum.

One suitable delay and sum back projection algorithm can be found in the [99]. Of course, other image reconstruction techniques can be used as well.

It will also be appreciated that there has been illustrated and described an arrangement where the photo-acoustic imaging apparatus 300 is arranged for the photo-acoustic transducer 320 to sense induced photo-acoustic signals 404 at plural points 406 along the curvilinear path 402 and, optionally, to perform signal averaging of the sensed induced photo-acoustic signals.

It is to be noted that volume 346 may be defined in other ways. For instance, the vertical lines 344 may, instead, be defined by the members 330, 334 of the photo-acoustic transducer holder. To give just one other example, the volume 346 could be defined by the support legs of the support frame 302. What is significant, allowing realisation of the benefits mentioned above, is the provision of the pulsed laser 310 as an integral part of the apparatus.

It is also to be noted that the horizontal dashed lines 344 defining the volume 346 may be provided at different levels.

Furthermore, it is not necessary to define the volume 346 as a regular, in this example cylindrical, volume.

In at least one arrangement, apparatus 300 is configured to vary the distance 347 between the pulsed laser 310 and the subject 306, or the region of interest 318 thereof. This may be done either by allowing the pulsed laser 310 to vary its position along the length of the pulsed laser mount 312, or for the length of the pulsed laser mount 312 to be varied/variable. Additionally or alternatively, holder 304 may be arranged such that its position with respect to the pulsed laser 310 or at least the position of the subject 306 (and/or the scanning area 316) may be varied. Therefore, photo-acoustic imaging apparatus 300 is arranged for a distance 346 between the light source 310 and the region 318 of the subject 306 to be selectively variable. As such, this provides the benefit that the radiation intensity in the region under image may be varied. Additionally or alternatively, the distance may be varied according to the properties of the subject 306.

As discussed above, and as illustrated in FIGS. 3(a)-(c), the photo-acoustic transducer 320 may be arranged so that it “faces” the scanning area 316. In the alternative arrangement of FIG. 3(d), this is not required. As can be seen from the figure, scanning plate 322 is mounted as before on the central axis 308 of the photo-acoustic sensing apparatus 300. A vertical support member 330 depends from, and is mounted on/to, scanning plate 322. However, in this arrangement, the photo-acoustic transducer 320 is not required to face the scanning area 316. In this arrangement, a member 348 is provided to “reflect” induced photo-acoustic signals into the field of view 338 of the photo-acoustic transducer 320, and sensing component 336. While FIG. 3(d) shows photo-acoustic transducer 320 being disposed in a vertical arrangement (the vertical member 330 is vertically aligned in an axis parallel to central axis 308), other alignments are also contemplated. As long as the reflecting member 348 is able to be positioned in order to reflect induced photo-acoustic signals into the field of view 338 and/or to sensing component 336, that will be sufficient. A significant idea behind the arrangement is that this member 348 acts as a guide member for induced photo-acoustic signals to be guided to the photo-acoustic transducer. In this example, the induced photo-acoustic signals are guided by means of being reflected to the photo-acoustic transducer and its sensing component.

In at least one arrangement, guiding/reflecting member 348 comprises a plate mounted on a part of the photo-acoustic transducer 320. A preferred angle of reflection is 45° from the horizontal/vertical axis. Optionally, reflecting member 348 may be pivotably mounted in order to vary the angle of reflection for the photo-acoustic signals to sensing component 336 to cater for on-site set-up requirements.

One particularly suitable reflector is the acoustic reflector F102, 45° reflector from OlympusNDT. This is made out of Type 303 stainless steel with surface finishes of 32 micro-inches.

Such arrangement(s) provides additional advantages. The design further reduces the size and weight of the photo-acoustic sensing apparatus 300. For instance, the member 348 may be composed of a material which is inherently lighter than the transducer holder members of FIG. 3(a). The design reduces the load on the driving motor 340 which may allow use of a motor having high-speed and will-load capacity.

Further, the design is considered simpler, thereby reducing manufacturing difficulties involved in producing the, for example, slightly more complex mounting arrangement of FIG. 3(a) with the photo-acoustic transducer holder members 330, 332, 334 illustrated therein. This also reduces the scanning radius required for the transducer and, as a result, it reduces the size of the ultrasound coupling medium tank used. This leads to an even more compact photo-acoustic tomography scanner with smaller tank. The tank size can be reduced by up to, say, 40% (e.g. a 40 cm×40 cm tank can be reduced to 25 cm×25 cm).

FIGS. 3(e) and 3(f) illustrate the point. If the photo-acoustic transducer is disposed generally in the horizontal plane as shown in FIG. 3(e), then this requires a relatively wide scanning radius as indicated by the arrow 350. However, in the alternative arrangement of FIG. 3(d), shown again in FIG. 3(f), when the photo-acoustic transducer 320 is in an arrangement outside of the horizontal plane, such as in or near the vertical plane as in FIG. 3(f), the scanning radius is reduced, thereby reducing the overall footprint of the device.

Referring again to FIG. 4, it will be appreciated that the curvilinear path 402 has a radius 408 which may be defined as originating at the central axis 308 of apparatus 300. In at least one arrangement, photo-acoustic apparatus 300 is configured for radius 408 to be selectively variable. Therefore, this allows for the photo-acoustic sensor to be moved according to the requirements of each set up to vary the scan radius (typically between 3 and 12 cm or thereabouts), and if necessary, the volume within which the pulsed laser diode is situated may be varied.

For comparative study, the pulsed laser 310 was removed from the photo-acoustic apparatus 300 and an OPO laser was incorporated as shown schematically in verification arrangement 500 of FIG. 5 The excitation laser comprises a 532 nm Nd:YAG laser 502 (Continuum, Surelite Ex) pumping an optical parametric oscillator 504 (Continuum, Surelite OPO) system). The OPO 504 generates 5 ns duration pulses at 10 Hz repetition rate with wavelength tunable from 680 nm to 2500 nm. A 590 nm long-pass filter (LGL590, Thorlabs) 506 is provided in front of OPO 504 filters the residual 532 nm beam. Then the 803 nm beam which is passed by filter 506 is reflected by a first antireflection coated right angle prism 508 through a first convex lens, routed to second antireflection coated right angle prism 512 through second convex lens 514 to antireflection coated right angle prism 516. The optical circuit is completed by a concave lens 518 which diverges the laser light 314, after it is passed through ground glass 520 which helps to improve the uniformity of the laser beam. Typically, the OPO beam has high divergence (>10 mrad), so one needs to use a lens collimator or long-focal length lenses to deliver the light from OPO to the sample. The laser fluence on sample surface is ^{˜}10 mJ/cm². Photo-acoustic signals are processed by processing unit 522 and digitised and recorded by computing device 524.

FIG. 6 shows an arrangement whereby the signals acquired by photoacoustic sensor 320 are processed. In this arrangement, a laser driver unit 600 drives the pulsed laser 310, thereby to generate laser light 314. The photo-acoustic signals picked up by photo-acoustic sensor 320 are routed to signal receiver, amplifier and filter circuitry 602. The conditioned signals are then processed by processor 604 which, in turn, controls the laser driver 600 and the drive motor 340.

Laser safety is of course an issue, although it is possible to mitigate any such concerns. When photo-acoustic tomography is used to image subjects in vivo, the maximum permissible pulse energy and the maximum permissible pulse repetition rate are governed by the ANSI laser safety standards [34]. The safety limits for the skin depend on the optical wavelength, pulse duration, exposure duration, and exposure aperture. In the spectral region of 700-1050 nm, the maximum permissible exposure (MPE) on the skin surface by any single laser pulse should not exceed 20×10^{2(λ-700)/1000} mJ/cm² (λ is the wavelength in nm) [34]. Therefore, at 803 nm the MPE is ^{˜}31 mJ/cm². The MPE for exposure time t=3 sec is 1.1×10^{2(λ-700)/1000}×t^{0.25} J/cm² (=1.1×10^{2(803-700)/1000}×3^{0.25} J/cm²=2.3 J/cm²) [34]. Since the pulsed laser 310 is operating at 7 kHz (total number of laser pulses=3×7000=21000), the MPE becomes 0.11 mJ/cm² (2.3/21000) per pulse. In one set up for photo-acoustic apparatus 300, the pulsed diode laser provides ^{˜}1.4 mJ pulse energy and the laser beam spread over an area ^{˜}5 cm² (^{˜}1.8 cm×2.8 cm). Therefore, the laser fluence is ^{˜}0.28 mJ/cm².

For an OPO-PAT system, the OPO has a laser energy output of ^{˜}100 mJ per pulse at 803 nm. However, by the time it reaches the sample surface some of its energy is lost as it has traversed several optical components (even after using IR coated optical components). Before the ground glass, the laser energy is ^{˜}80 mJ per pulse. The beam is then spread over an area of ^{˜}8 cm² after the ground glass and thus on the sample the fluence is measure to be 10 mJ/cm². This is within the MPE safety limit. Since the OPO is running at 10 Hz, for long exposure (>10 s), The MPE should be within 200 mW/cm² or 20 mJ/cm² (200 mW/10 Hz=20 mJ) per pulse. Therefore, the OPO laser energy on the sample surface is within the safety limit.

When imaging phantoms it is not strictly necessary to follow the MPE safety limit using a pulsed laser system such as that of FIG. 3; this setup may be used to determine the optimal performance for a pulsed laser system, and what is feasible in terms of maximum energy and maximum repetition rate. As mentioned above, any laser safety issues can be mitigated. For in vivo operation, the fluence can be reduced by spreading the beam over a larger area or reducing the pulse repetition rate or reducing the laser power output by controlling the power supply itself. For example, the MPE of a pulsed laser photo-acoustic tomography system can be made 0.28 mJ/cm² by operating the pulsed laser at ^{˜}2740 Hz, while still setting the scan time to be three seconds. Another way is to make the scan faster by using multiple, i.e. N, photo-acoustic sensors and instead of conveying one photo-acoustic sensor along the full curvilinear path, conveying the multiple sensors each one nth part of the full curvilinear path. So, where the curvilinear path is a circular path such as that illustrated in FIGS. 3 and 4, this would mean that, while rotating the scanning plate 322 in a full circle of 360°, it is necessary only to rotate each photo-acoustic transducer 360/N degrees, or a part thereof. Once the total scan time is reduced, the MPE limit will also change. That is, photo-acoustic imaging apparatus 300 comprises a plural number of photo-acoustic transducers 320, wherein a distance of travel of each of the plural photo-acoustic transducers along the curvilinear path is defined as a full distance of the curvilinear path modified by the plural number of photo-acoustic transducers. In this instance, the “modification” is a division. The distance of travel of one photo-acoustic sensor is calculated as the full length of the curvilinear path divided by the number of photo-acoustic sensors.

Thus, an affordable and compact PLD-PAT device for high-speed PAT imaging is demonstrated. The performances of PLD-PAT and OPO-PAT systems are compared. The PLD-PAT could provide A-line data in scan time 3 s to form a 2D image with good SNR (^{˜}30). 2-cm deep tissue images with SNR 10 in 30 s scan time was possible with PLD-PAT. In spite of having almost 70 times less optical energy output per pulse, the PLD-PAT system can provide an alternate solution for low-cost, light weight and portable, real-time PAT imaging with single-element transducer. The imaging depth can be enhanced further by usage of various photoacoustic contrast agents reported widely in the literature for NIR wavelength range [35-37]. The imaging speed can further be improved by using multiple ultrasound transducers at the same time. In this work, the conclusions are drawn based on the results obtained on phantoms only. To demonstrate the potentiality of PLD-PAT for high-speed and deep-imaging for in vivo applications, it is possible to work on in vivo imaging of small animal brain. The portability, the low-cost, and the image quality at high-speed promises that the proposed PLD-PAT system will find applications in biomedical imaging applications.

### Experimental Data 1—Horse-Hair Phantom Imaging

The photograph of horse-hair phantom prepared is shown in FIG. 7(a). Here the horse-hair was glued on plastic tubes. The hair has a diameter of ^{˜}100 μm. The images of hair phantom obtained by collecting photo-acoustic signals in 30, 20, 10, 5 and 3 sec scan time using the pulsed laser photo-acoustic tomography techniques described above are shown in FIGS. 7(b-f) with a single 2.25 MHz UST. FIGS. 7(g-m) show the images of the same phantom obtained by collecting photo-acoustic signals in 120, 60, 30, 20, 10, 5 and 3 sec scan time using OPO-PAT with a single 2.25 MHz UST. The line profiles along A, B [indicated on FIGS. 7(b) and 7(i)] are compared in FIG. 7(n). To study the effect of scan speed on the quality of image, the signal-to-noise ratio (SNR) of images acquired at different scan speeds were calculated. The signal-to-noise ratio (SNR) is defined as the peak-to-peak amplitude of the photo-acoustic signal divided by the standard deviation of the noise, SNR=V/n, here V is the peak-to-peak for two-acoustic signal amplitude, and n is the standard deviation of the background noise. The SNR as a function of scan time is plotted for both the PLD-PAT and OPO-PAT systems shown in FIG. 7(o). At 3 sec scan time, PLD-PAT can provide image with SNR ^{˜}29, one can achieve same SNR in OPO-PAT at 30 s. To increase the imaging speed further multiple photo-acoustic sensors (e.g. ultrasonic transducers) can be used, as described above. The circular scanner was designed to mount multiple photo-acoustic sensors simultaneously to make the data collection much faster. If ‘N’ photo-acoustic sensors are mounted, it can reduce the data acquisition time up to ^{˜}(3/N) sec, as also mentioned above. It was also demonstrated that photo-acoustic signals obtained by scanning in 180 degree would be sufficient to reconstruct the object with appropriate modified reconstruction technique [9]. Thus, with improved reconstruction of partial data acquisition the scan time can be reduced to ^{˜}(3/2 N) sec. Therefore, if 4 USTs are used, the system can work at ^{˜}2.6 Hz frame rate. The above experiments are repeated for a single-element photo-acoustic sensor 5 MHz, and the results are shown in FIG. 8. The images from the 2.25 MHz photo-acoustic sensor have better SNR because there is more photo-acoustic energy in the low-frequency range, hence it can receive a stronger signal than the other high central frequency transducer. But the images from a 5 MHz ultrasonic transducer are sharper as expected. From FIGS. 7n and 8m, the spatial resolution values measured from the FWHM values are ^{˜}380 μm and ^{˜}180 μm for 2.25 MHz and 5 MHz UST, respectively. From FIGS. 7(o) and 8(n), the SNR of the reconstructed images increases with scan time for both the transducers because increasing the scan time increases the number of recorded signals (A-lines).

From the results obtained using the horse hair phantom, some conclusions may be drawn. First of all, the energy of the pulsed laser is almost ^{˜}70 times weaker, however, due to the higher repetition rate, the low energy is compensated by more numbers of A-line signals. Therefore, in comparison with an OPO-PAT system the performance of the pulsed laser system is very impressive. For example, a pulsed laser diode has a 136 ns pulse width compared to 5 ns for the OPO laser. However, for photo-acoustic tomography imaging this pulse width will have no effect. The spatial resolution as calculated from both the systems matches very well. Since PAT typically uses low frequency ultrasound transducers (1-5 MHz), the longer pulse width of the pulsed laser diode will have no effect on the cross-sectional imaging. Lastly, due to higher repetition rate a pulsed laser system is capable of very fast imaging. As shown, even in 3 sec scanning acceptable photo-acoustic tomography images were obtained. Using traditional OPO lasers such imaging speed cannot be obtained using single photo-acoustic sensor (ultrasonic transducer) scanning.

### Experimental Data 2—Imaging Inside Chicken Breast Tissue

Deep tissue imaging experiments were carried out on low-density polyethylene (LDPE) tube (^{˜}10 mm long and ^{˜}0.6 mm inner diameter), filled with mice blood. The LDPE tube was placed on a chicken breast tissue as shown in FIG. 9(a). For imaging they were covered by tissues of various thicknesses as shown in FIG. 9(b). The tissue cross-section containing the LDPE tubes was imaged when tissue slices were sequentially placed to make the tubes 1 cm, and 2 cm, 3 cm deep from laser-illuminated tissue surface. PAT images were acquired using 2.25 MHz at 30 s, 20 s, 10 s, 5 s, and 3 s scan time. FIGS. 9c and 9e show the PAT images acquired at 1 cm, and 2 cm depth, using PLD-PAT, respectively. The blood image at 2 cm depth obtained in 3 s has good contrast. FIGS. 9d, 9f, and 9g show the PAT images acquired at 1 cm, 2 cm, and 3 cm depth using OPO-PAT, respectively. Only the image at 30 s has good quality. In FIG. 9h, the SNR of blood embedded in tissue images obtained in 30 s using PLD-PAT and OPO-PAT systems was compared.

Deep imaging experiments were also carried out on two LDPE tubes, one filled with mice blood and other filled with ICG. The ICG solution was prepared with 323 μM concentration to have an absorption peak ^{˜}800 nm. The tissue cross-section containing the LDPE tubes was imaged when tissue slices were sequentially placed to make the tubes 1 cm, and 2 cm deep from laser-illuminated tissue surface. PAT images were acquired using 2.25 MHz at 5 sec, and 3 sec scan time using the PLD-PAT system only. FIGS. 10 a, b and 10 c, d show the PAT images acquired at 1 cm, and 2 cm depth, respectively. The SNR values of blood, ICG measured from FIG. 10 b are ^{˜}18, ^{˜}23 and that measured from FIG. 10 d are ^{˜}6, ^{˜}10, respectively. Both the tubes were clearly visible even at 2 cm under the chicken breast tissues.

The performance of PLD-PAT in comparison with the OPO-PAT systems are summarised in Table 1. From the hair phantom and the tissue imaging it is evident that the OPO-PAT was able to provide acceptable imaging in 30 s and imaging depth 3-cm. Whereas PLD-PAT system can obtain acceptable image in 3 s. Although, PLD has low pulse energy, up to 2 cm imaging depth was obtained with good SNR, thus making it particularly suitable for biomedical imaging applications with such an imaging depth. Also the system is capable of providing volumetric images of the sample. The sample may be scanned along z-axis using a motorised/manual mechanical stage. The low pulse energy was slightly compensated by the higher pulse repetition rate. Because of which even within 3 sec scan time enough numbers of A-lines were collected to give acceptable PAT images. Moreover, since the PLD was integrated inside the scanner, there were minimum losses in the laser energy from the laser source to the sample. For traditional OPO laser one needs to have a light delivery system (fibre optics or free-space optics). In both the cases there will be a significant amount of energy loss ^{˜}25-30%. However, the current PLD systems have few drawbacks which can be improved: (a) low pulse energy on the tissue surface (^{˜}10 times lower than OPO), high energy PLDs can be obtained in the near future to improve the imaging depth, (b) generates rectangular beam with stripes, it can be improved using micro-optics, low-diffusive ground glass, etc. (c) PLD is not a tunable source yet, but multiple wavelength PLDs can be obtained in the near future for spectroscopic imaging.

In summary, in spite of having few drawbacks of the PLDs, it is expected that pulsed laser foot-acoustic tomography techniques will find strong interest from the imaging community, due to its compactness (no need for optical table, portable), less cost (4-5 times cheaper than traditional OPO lasers), fast imaging capability, decent imaging depth (2 cm).

TABLE 1
Comparison of various parameters between PLD-PAT and OPO-PAT.

| Sl No. | Parameter | | PLD-PAT | OPO-PAT |
|---|---|---|---|---|
| 1 | Spatial | 2.25 MHz | 384 μm | 381 μm |
| | resolution | 5 MHz | 185 μm | 182 μm |
| | (FWHM) | | | |
| 2 | SNR | 2.25 MHz | 29 (3 s) & | 28 (30 s) |
| | | | 84 (30 s) | |
| | | 5 MHz | 24 (3 s) & | 23 (30 s) |
| | | | 77 (30 s) | |
| 3 | Imaging speed | | Acceptable | Acceptable image in |
| | | | image in 3 s | 30 s |
| 4 | Imaging depth | | ~2 cm (SNR | ~3 cm (SNR 3 at 30 s) |
| | | | 10 at 30 s) | ~4 cm (120 s scanning |
| | | | | time) |
| 5 | Image | 3 s | Acceptable | Not Acceptable |
| | quality | 30 s | Good | Good |

| | | | |
|---|---|---|---|
| 6 | Pulse duration | ~136 ns | ~5 ns |
| 7 | Repetition rate | 7 kHz | 10 Hz |
| 8 | Pulse energy per pulse | 1.4 mJ at laser | 100 mJ at laser output |
| | | output | 10 mJ/cm² (at sample) |
| | | 0.28 mJ/cm² | |
| | | (at sample) | |
| 9 | Laser head dimensions | 11.0 × 6.0 × | 77.5 × 17.8 × 19.0 cm |
| | | 3.6 cm | |
| 10 | Laser head weight | ~150 gm | ~100 Kg |
| 11 | Portability | Yes | No (optical table |
| | | | needed) |
| 12 | Cost | ~15-25k USD | ~90-140k USD |

### Experimental Data 3—Experiments Blood/Ink

A first experiment in this respect was conducted on a blood/ink sample. A low density polyethylene (LDPE) tube (inner diameter: 0.59 mm) filled with black ink and an ultrasonic transducer were mounted inside water as described above. The tube was placed at ^{˜}4 cm distance from the laser window. The photo-acoustic signal received by the ultrasonic transducer was band pass filtered (1-10 MHz) and amplified with 50 dB gain. Finally, the signal was digitised by a DAQ card at 50 Ms/s and stored in computer. A total of 7000 A-lines (1 sec) were collected. Similarly, photo-acoustic signals from blood were also acquired. To confirm that the LDPE tube has no contribution to the photo-acoustic signals, the experiment was repeated with an unfilled LDPE tube as well. FIG. 11 shows the photo-acoustic signals averaged 700 times (0.1 sec). The photo-acoustic signals obtained with single pulse excitation (no averaging) are also shown as profiles A, B, C, and D in FIG. 11 (inset). The photo-acoustic signal generated by black ink is as strong as that generated by blood which indicates that they have similar optical absorption coefficients at ^{˜}803 nm. At 800 nm, the absorption coefficient for whole blood is ^{˜}5 cm−1.

A second experiment was conducted on blood/ink embedded inside chicken breast tissue. The LDPE tube filled with black ink or blood was embedded in the chicken breast tissue (CBT). The tube was still kept the same distance 4 cm from the laser window. Cut pieces of chicken breast tissue having thicknesses of 2, 4, 6 cm were used. The LDPE tube was embedded in the middle of the tissue sample. Photo-acoustic signals were collected when the tube was placed at 1, 2, or 3 cm deep from the laser illuminated tissue surface. The generated photo-acoustic signal also needs to travel 1, 2, or 3 cm inside the attenuating chicken breast tissue before it is received by the transducer. The incident laser energy density on the tissue surface area was ^{˜}0.3 mJ/cm², which is much less than the “maximum permissible exposure (MPE)” of 32 mJ/cm² at 803 nm [36-38]. FIGS. 12 (a) and (b) show the OA signals collected at these depths by the 2.25 and 5 MHz ultrasonic transducer, respectively. Similarly, the above experiment was repeated with LDPE tube filled with blood embedded inside the tissue. FIGS. 12 (c) and (d) show the PA signals from blood collected by the 2.25 and 5 MHz UST, respectively. In FIG. 12, each signal was averaged 700 times. In our current experiments, both black ink and blood were successfully detected in chicken breast tissue at depth of ^{˜}3.0 cm.

For the two experiments, the signal-to-noise ratio (SNR) was calculated using the relation, SNR=V/n, where V is the peak-to-peak PA signal voltage and n is the standard deviation of the background noise. The SNR in terms of dB can be expressed as SNR(dB)=20×(log)₁₀ (V/n). The SNR as a function of square root of number of signal averaging (VN) for a 5 MHz ultrasonic transducer is shown in FIG. 13 (a). The graph clearly shows the improvement in SNR with increase of N. FIG. 13 (b) shows the SNR versus penetration depth (D) inside the chicken breast tissue. Due to the overwhelming light scattering in the chicken tissue, the intensity of light, and hence the SNR decreased with increasing depth. The maximum penetration depth measured with black ink or blood in chicken breast tissue was ^{˜}3.0 cm. The SNR at this depth reached ^{˜}15 for black ink and ^{˜}10 for blood, after averaging 700 times.

### Experimental Data 4—Pulsed Laser Diode Photo-Acoustic Tomography Imaging

The good signal strength and SNR of the photo-acoustic signals generated using the pulsed laser diode allowed imaging. For photo-acoustic imaging the continuous scan [39] technique was used in which in which the stepper motor rotates the ultrasonic transducer (UST) continuously at a predefined speed, collecting signals while moving. In this arrangement, the A-lines were saved once the rotation is complete. Imaging experiments were performed on two different samples. Sample-1 is horse hair phantom prepared in a triangular shape as shown in FIG. 14 (a). Here the horse-hair was glued on plastic tubes. The hair has side-length of ^{˜}8 mm and diameter of ^{˜}150 μm. The images of hair phantom obtained by collecting photo-acoustic signals in 20, and 10 sec scan time using 2.25 MHz UST (FIGS. 14 b, 14 c) and 5 MHz UST (FIGS. 14, 14 e). The line profiles along A, B [indicated on FIGS. 14 (b) and 14 (d)] are compared in FIG. 14 (f). The full width at half-maximum (FWHM) values are ^{˜}435 μm and ^{˜}285 μm for the 2.25 MHz and 5 MHz transducer is, respectively. To study the effect of scan speed on the quality of image, the SNR values of images acquired at different scan speeds were calculated. The SNR values are 175 and 130 (with the 2.25 MHz transducer), 160 and 115 (with the 5 MHz transducer) for images in FIGS. 14b, 14c and 14d, 14e, respectively. The images from the 2.25 MHz UST have better SNR because there is more photo-acoustic energy in low-frequency range, hence it can receive stronger signal than other transducer. But the images from the 5 MHz UST are sharper (higher spatial resolution). Reducing the scan time reduces the number of recorded signals (A-lines) and hence, the SNR of the reconstructed images reduces with scan time for both the transducers. For the 2.25 MHz UST, reducing the scan time from 20 to 10 s, reduces the SNR from 175 to 130. However, the PLD-OAT system promises to provide good image quality even at high scan speed of 10 s. The Nd:YAG laser based PAT systems will take several minutes for A-line collection, due to low-repetition rate, to generate a good quality image.

Deep-tissue imaging experiments were carried out on another sample, “sample two”, which is made of two LDPE tubes (^{˜}12 mm long and ^{˜}0.59 mm inner diameter), one filled with mice blood and other filled with indocyanine green (ICG). The two LDPE tubes were placed on a chicken breast tissue as shown in FIG. 15 (a). For imaging they were covered by tissues of various thicknesses as shown in FIG. 15 (b). The ICG solution was prepared with 323 μM concentration to have an absorption peak ^{˜}800 nm. The tissue cross-section containing the LDPE tubes was imaged when tissue slices were sequentially placed to make the tubes 1 cm, and 2 cm deep from laser-illuminated tissue surface. OAT images were acquired on “Sample 2” using the 2.25 MHz UST at 20 sec and that 10 sec scan time. FIGS. 15(c, d) and 15(e, f) show the photo-acoustic tomography images acquired at 1 cm, and 2 cm depth, respectively. The SNR values of blood, ICG measured at 1 cm are ^{˜}25, ^{˜}32 and that measured at 2 cm are ^{˜}8, ^{˜}14, respectively. Both the tubes were clearly visible even at 2 cm under the chicken breast tissues. Both the tubes were clearly visible even at 2 cm under the chicken breast tissues.

### Experimental Data 5—Experiments with Mouse Blood and Ink

A NIR pulsed diode laser (Quantel DQ-Q1910-SA-TEC) of ^{˜}803 nm wavelength, pulse energy ^{˜}1.45 mJ per pulse at a very high pulse repetition rate of 7 kHz was used as the photo-acoustic excitation source. The laser is capable of producing ^{˜}136 nano second pulses. A 2.25 MHz center frequency 13 mm active area diameter nonfocused ultrasound transducer (UST) was used for the detection of the photo-acoustic signal. To synchronize the acquisition of the data, same function generator (HTRONIC FG 250D) was used to trigger both the laser as well as the data acquisition system. The photo-acoustic signal was first amplified with a pulse/receiver amplifier (Olympus, 5072PR) and then digitized and recorded using a data acquisition card (Gage, CompuScope 4227) connected with a desktop computer. Experiments were performed on mouse blood/ink sample inside LDPE tube (inner diameter: 590 μm, wall thickness: 190 μm). Two LDPE tubes, one filled with black ink (Parker, France) and the other one filled with mouse blood were prepared for this experiment. The transducer and the LDPE tubes were mounted in a transparent container (made out of Perspex) filled with water. The tube, placed at ^{˜}4 cm distance from the laser window, was irradiated with pulse energy density of ^{˜}0.85 mJ/cm² in the beam area 2×0.85 cm². The photo-acoustic signal received by the ultrasonic transducer was band pass filtered (1-10 MHz) and amplified with 50 dB gain. Finally, the signal was digitized by a DAQ card at 50 Ms/s and stored in the computer. A total of 14,000 A-lines (2 sec) were collected. To measure the penetration-depth capabilities of the system, LDPE tube filled with black ink or blood was embedded in the chicken breast tissue (CBT). The tube was still kept the same distance 4 cm from the laser window. The LDPE tube was embedded in the middle of the tissue sample. Photo-acoustic signals were collected when the tube was placed at 1, 2, or 3 cm deep from the laser illuminated tissue surface. The generated PA signal also needs to travel 1, 2, or 3 cm inside the attenuating chicken breast tissue before it is received by the transducer. FIG. 16a shows the photo-acoustic signals averaged 700 times (0.1 sec) of the black ink. It is possible to see clearly the photo-acoustic signal generated from up to a depth of 3 cm inside chicken breast tissue. Of course with increase in depth the PA signal amplitude drops. In the inset the PA signal from the ink tube in water is shown. FIG. 16b shows the similar experimental data but with blood filled tube. The photo-acoustic signal generated by black ink is as strong as that generated by blood which indicates that they have similar optical absorption coefficients at ^{˜}803 nm.

The photoacoustic signal in the chicken breast tissue sample was very encouraging. Therefore, the experiment was continued to see if deep-tissue imaging can also be done. For the imaging a photoacoustic tomography system in the orthogonal illumination mode was used. Orthogonal photoacoustic tomography is known for deep-tissue imaging. In photo-acoustic tomography the detector is rotated around the sample using a stepper motor (Lin Engineering, Silverpak 23C) and a mechanical scanner. A simple MATLAB based program was used to control the data acquisition, stepper motor motion and also the reconstruction of the photo-acoustic data. FIG. 17a shows the tissue phantom used to study the imaging performance of the system. For photo-acoustic tomography typically low frequency transducers are used, as they are best suited for deep tissue imaging. Therefore in this study the same 2.25 MHz center frequency non-focused 13 mm active area diameter detector was used. Two LDPE tubes (length ^{˜}10 mm), one filled with mouse blood and the other ICG (indocyanine green) was placed on top of the chicken breast tissue and it was covered with another layer of chicken breast tissue with thickness 1 and 2 cm for two experiments. ICG solution was prepared to have absorption peak around ^{˜}800 nm wavelength light.

Various imaging speeds were tested by controlling the transducer rotation speed. 10 second, and 20 seconds imaging speeds were tested. FIGS. 17b and 17c show reconstructed PAT images of the cross-sectional image of the phantom for 20 sec and 10 sec scanning speed, respectively, when the tubes are buried inside 1 cm thick chicken breast tissue. FIGS. 17d and 17e show reconstructed PAT images of the cross-sectional image of the phantom for 20 sec and 10 sec scanning speed, respectively, when the tubes are buried inside 2 cm thick chicken breast tissue. All our reconstruction was done using a simple delay-and-sum back projection reconstruction algorithm in MATLAB.

It is clearly evident from the FIG. 17 that even with 2 cm deep-tissue it was possible to reconstruct back the optical absorption map using PAT with reasonable SNR and resolution. Most traditional PAT systems use bulky Nd:YAG pump lasers with high pulse energy. However, with portable and small pulsed diode laser also it was possible to image 2 cm deep inside biological tissue. The imaging speed is also improved over other existing PAT systems. For certain applications, the image resolution obtained with the short time scanning is just good enough and would be sufficient. Traditional lasers used for PAT has a pulse repetition rate in the order of 10-20 Hz. As a result to collect enough number of photo-acoustic signals around the object the transducers need to rotate the sample slowly. As a result the image acquisition time is quite slow. Typically several minutes are needed for full rotation. However, with the use of high repetition rate pulsed diode laser it is possible to collect data very fast (10 s) and still obtain a very good quality PAT image. This is close to 10 fold improvement in terms of imaging speed.

### Experimental Data 6—Human Hair Phantom

A NIR pulsed diode laser (Quantel DQ-Q1910-SA-TEC) of ^{˜}803 nm wavelength, pulse energy ^{˜}1.45 mJ per pulse at a very high pulse repetition rate of 7 kHz illuminates the sample from above. The laser is capable of producing ^{˜}136 nano second pulses. The sample is placed inside a water bath. A 5 MHz/2.25 MHz centre frequency ultrasound transducer for the detection of the signal was used. To synchronize the acquisition of the data, same function generator (HTRONIC FG 250D) was used to trigger both the laser as well as the data acquisition system. The photo-acoustic signal was first amplified with a pulse/receiver amplifier (Olympus, 5072PR) and then digitized and recorded using a data acquisition card (Gage, CompuScope 4227) connected with a desktop computer. The detector is rotated around the sample using a stepper motor (Lin Engineering, Silverpak 23C) and a mechanical scanner. A simple MATLAB based program was used to control the data acquisition, stepper motor motion and also the reconstruction of the photo-acoustic data. A phantom (a black human hair cross) was used to study the imaging performance of the system. The human hair is ^{˜}50-75 micron diameter. Different speeds of transducer rotation were tried in an attempt to observe the least possible time in which to obtain an image with a good SNR and resolution.

For photo-acoustic tomography typically low frequency transducers are used, as they are best suited for deep tissue imaging. Therefore in this study 5 and 2.25 MHz centre frequency non-focused 13 mm active area diameter ultrasound detectors were used. Moreover, the laser pulse width is ^{˜}136 ns, which results roughly a maximum bandwidth of ^{˜}6.5 MHz of PA signals. Therefore, using a higher centre frequency than this will result sub-optimal detection of the PA signals. On the other hand if very low frequency transducers are used, that will reduce the spatial resolution of the imaging system. The spatial resolution of the photo-acoustic tomography reconstructed images is roughly related to wavelength of the detected ultrasound. For 5 MHz ultrasound the wavelength is ^{˜}300 micron. Therefore, it is possible to achieve ^{˜}150 micron spatial resolution with 5 MHz transducer. Similarly, with 2.25 MHz detector it is possible to achieve ^{˜}300 micron spatial resolution.

Various imaging speeds were tested by controlling the transducer rotation speed. 5 seconds, 10 second, 20 seconds, and 30 seconds imaging speeds were tested. FIG. 18 shows reconstructed photo-acoustic tomography images of the cross-sectional image of the phantom for various imaging speed for 2.25 MHz detector. FIG. 19 shows similar images but with 5 MHz detector. All our reconstruction was done using a simple delay-and-sum back projection reconstruction algorithm in MATLAB.

It is evident from the FIGS. 18 and 19 that even with 5 s imaging speed it is possible to recover the cross-sectional images with high SNR. FIG. 20 shows the SNR vs scanning time. The SNR is calculated from the reconstructed image using the following formula: SNR=A_signal/σ_noise, where A_signal is the amplitude of the signal on the hair, and σ_noise is the standard deviation of the noise from the background. Of course with higher scan time the SNR will improve as expected. But even with 5 s scan the SNR is good enough for in vivo imaging application in the future. Moreover, even with 5 seconds rotation speed of the transducer the obtained image shows the structural features with a clarity which is not very different from the typical 30 seconds scanning speed. As expected with the higher frequency detector, one can achieve high resolution imaging as seen between FIG. 18(a) and FIG. 19(a). For certain applications, the image resolution obtained with the short time scanning is just good enough and would be sufficient. The imaging was done with very fine hair structure and the images had high spatial resolution and hence it is believed it is possible to visualise the ultrafine blood vessels in the body in a faster and cheaper way.

Traditional lasers used for photo-acoustic tomography have a pulse repetition rate in the order of 10-20 Hz. As a result to collect enough number of photo-acoustic signals around the object the transducers need to rotate the sample slowly. As a result the image acquisition time is quite slow. Typically several minutes are needed for full rotation. However, with the use of high repetition rate pulsed diode laser it is possible to collect data very quickly (5 s) and still obtain a very good quality PAT image. This is close to 10-20 fold improvement in terms of imaging speed. From all the reconstructed images it is possible to see a striated pattern in the reconstructed images. It is due to the laser beam profile. The pulse diode laser beam profile is striated and not completely homogeneous. Therefore, it is getting reflected in the reconstructed image. However, during in vivo study it will not be a problem. Since during in vivo application light has to penetrate the tissue and will get scattered. Thus the prominence of the striated pattern will reduce.

The imaging speed can further be improved by using multiple detectors. For example it is possible to use 4-8 detectors and data can be collected in parallel. Thus another factor of 8 improvements is feasible. Thus it is possible to obtain PAT images less than one second scan-time. That way real-time PAT imaging is possible and several dynamic study can be done.

### Experimental Data 7—Phantoms and Blood Embedded Inside Biological Tissues

FIG. 21 shows in vivo photo-acoustic tomography imaging of a mouse brain in a lateral playing at different scanning speeds acquired using a 2.25 MHz ultrasonic transducer. The images are, respectively, (a) a 30 second scan time, (b) a 20 second scan time, (c) a 10 second scan time, (d) a five second scan time and (e) a three second scan time. FIG. 21 (f) illustrates the signal to noise ratio as a function of scan time where SS—Superior sagittal sinus, TS—Transverse Sinus.

FIG. 22 illustrates hair phantom images of a pulse laser diode photo-acoustic tomography imaging system (a-c) and an OPO-PAT (d-f) at different speeds. FIG. 22(g) illustrates the FWHM profile and FIG. 22(h) illustrates the signal-to-noise ratio plots for OPO and PLD PAT systems.

In our current in vivo animal experiments, 2 male and 2 female healthy mice of body weight 28±3 gm and aged 6 weeks were used. All experiments were performed in accordance with the approved guidelines and regulations, and were approved by the institutional Animal Care and Use committee of Nanyang Technological University, Singapore (Animal Protocol Number ARF-SBS/NIE-A0263). For in vivo imaging the mouse was anesthetized. The anaesthetic cocktail contains Ketamine and Xylazine in the dosage of 120 mg/kg and 16 mg/kg respectively. 0.1 ml per 10 g of mouse body weight was injected intraperitoneally. Before imaging experiments, the hair on the head of the small animal was depilated using hair removal cream. The mouth and nose of the animal were covered with a breathing mask to deliver anaesthesia mixture. The anaesthesia was achieved by the inhalation of a mixture of O2 and isoflurane. A custom-designed animal holder was used to mount the animal. The animal was placed in sitting position, on its abdomen, and the body of the animal was secured to the mount with surgical tapes to provide grip the animal. During experiments, the animal and the animal holder were mounted on a translation stage to align the brain to the centre of the scanning geometry. After the data acquisition for PAT, the animal was sacrificed by the intraperitoneal injection of pentobarbital of concentration 300 mg/ml.

The brains of healthy mice were non-invasively imaged using our PLD-PAT system.

The mouse was placed at the centre of the circular scanning area and laser illumination area. The A-lines signals from the mouse brain were collected using a circular scanning single-element 2.25 MHz UST. The in vivo images acquired at different scan speeds using the PLD-PAT system are shown. In all the images the images the superior sagittal sinus (SS) and transverse sinuses (TS) of the mouse brain are clearly visible. The resolution of our PLD-PAT system with 2.25 MHz is ^{˜}380 μm, hence the superficial/bridging veins whose diameter is <200 μm are not clearly visible in the PAT images. To study the effect of scan speed on the quality of the in vivo images, the signal-to-noise ratio (SNR) of images acquired at different scan speeds was calculated. The SNR was defined as the peak-to-peak amplitude of the PA signal divided by the standard deviation of the noise, SNR=V/n, here V is the peak-to-peak PA signal amplitude, and n is the standard deviation of the background noise.

FIG. 23 illustrates images acquired using deep PLD-PAT imaging of ICG and blood at different speeds acquired using a 2.25 MHz ultrasonic transducer (a, b) 1 cm, (c, d) 2 cm.

An affordable and portable PLD-PAT system for high-speed in vivo imaging is demonstrated. The in vivo brain images acquired at different scan speeds are presented. The A-line data collected in 3 s could provide the reconstructed 2D image of lateral view of the brain. It is possible to use an optical contrast agent to improve the image contrast and imaging depth in our PLD-PAT system. The imaging speed can further be improved by using multiple ultrasound transducers at the same time. The portability, low-cost, image quality promises that the proposed system will find near-real time in vivo imaging applications in biomedical imaging areas

It will be appreciated that the invention has been described by way of example only and that various modifications may be made to the techniques described above without departing from the spirit and scope of the invention.

### REFERENCES

1. L. V. Wang, and S. Hu, “Photoacoustic Tomography: In Vivo Imaging from Organelles to Organs,” Science 335, 1458-1462 (2012).
2. M. Xu, and L. V. Wang, “Photoacoustic imaging in biomedicine,” Rev. Sci. Instrum. 77, 041101 (2006).
3. L. V. Wang, “Prospects of photoacoustic tomography,” Med. Phys. 35, 5758-5767 (2008).
4. C. Li, and L. V. Wang, “Photoacoustic tomography and sensing in biomedicine,” Phys. Med. Biol. 54, R59-97 (2009).
5. L. V. Wang, “Multiscale photoacoustic microscopy and computed tomography,” Nat Photonics 3, 503-509 (2009).
6. R. A. Kruger, R. B. Lam, D. R. Reinecke, S. P. Del Rio, and R. P. Doyle, “Photoacoustic angiography of the breast,” Med. Phys. 37, 6096 (2010).
7. P. Beard, “Biomedical photoacoustic imaging,” Interface Focus 1, 602-631 (2011).
8. M. Pramanik, G. Ku, C. Li, and L. V. Wang, “Design and evaluation of a novel breast cancer detection system combining both thermoacoustic (TA) and photoacoustic (PA) tomography,” Med. Phys. 35, 2218 (2008).
9. C. Huang, K. Wang, L. Nie, L. V. Wang, and M. A. Anastasio, “Full-Wave Iterative Image Reconstruction in Photoacoustic Tomography with Acoustically Inhomogeneous Media,” IEEE Trans. Med. Imaging 32, 1097-1110 (2013).
10. M. Xu, and L. V. Wang, “Universal back-projection algorithm for photoacoustic computed tomography,” Phys. Rev. E 71, 016706 (2005).
11. C. Lutzweiler, X. L. Dean-Ben, and D. Razansky, “Expediting model-based optoacoustic reconstructions with tomographic symmetries,” Med. Phys. 41, 013302 (2014).
12. J. Prakash, A. S. Raju, C. B. Shaw, M. Pramanik, and P. K. Yalavarthy, “Basis pursuit deconvolution for improving model-based reconstructed images in photoacoustic tomography,” Biomed. Opt. Express 5, 1363 (2014).
13. B. Lashkari, and A. Mandelis, “Comparison between pulsed laser and frequency-domain photoacoustic modalities: signal-to-noise ratio, contrast, resolution, and maximum depth detectivity,” Rev. Sci. Instrum. 82, 094903 (2011).
14. L. V. Wang, “Tutorial on Photoacoustic Microscopy and Computed Tomography,” IEEE Journal of selected topics in Quantum electronics 14, 171-180 (2008).
15. B. Dong, H. Li, Z. Zhang, K. Zhang, S. Chen, C. Sun, and H. F. Zhang, “Isometric multimodal photoacoustic microscopy based on optically transparent micro-ring ultrasonic detection,” Optica 2, 169 (2015).
16. X. Wang, J. B. Fowlkes, J. M. Cannata, C. Hu, and P. L. Carson, “Photoacoustic imaging with a commercial ultrasound system and a custom probe,” Ultrasound Med. Biol. 37, 484-492 (2011).
17. T. N. Erpelding, C. Kim, M. Pramanik, L. Jankovic, K. Maslov, Z. Guo, J. A. Margenthaler, M. D. Pashley, and L. V. Wang, “Sentinel Lymph Nodes in the Rat: Noninvasive Photoacoustic and US imaging with a clinical US system,” Radiology 256, 102-110 (2010).
18. W. Xia, D. Piras, M. K. A. Singh, J. C. G. van Hespen, T. G. van Leeuwen, W. Steenbergen, and S. Manohar, “Design and evaluation of a laboratory prototype system for 3D photoacoustic full breast tomography,” Biomed. Opt. Express 4, 2555-2569 (2013).
19. A. Buehler, X. L. Dean-Ben, J. Claussen, V. Ntziachristos, and D. Razansky, “Three-dimensional optoacoustic tomography at video rate,” Opt. Express 20, 22712-22719 (2012).
20. P. V. Es, S. K. Biswas, H. J. B. Moens, W. Steenbergen, and S. Manohar, “Initial results of finger imaging using photoacoustic computed tomography,” J. Biomed. Opt. 19, 060501 (2014).
21. C. Li, A. Aguirre, J. Gamelin, A. Maurudis, Q. Zhu, and L. V. Wang, “Real-time photoacoustic tomography of cortical hemodynamics in small animals,” J. Biomed. Opt. 15, 010509 (2010).
22. D. Piras, W. Steenbergen, T. G. van Leeuwen, and S. Manohar, “Photoacoustic Imaging of the Breast Using the Twente Photoacoustic Mammoscope: Present Status and Future Perspectives,” IEEE Journal of Selected Topics in Quantum Electronics 16, 730-739 (2010).
23. J. S. Allen, and P. Beard, “Pulsed near-infrared laser diode excitation system for biomedical photoacoustic imaging,” Opt. Lett. 31, 3462-3464 (2006).
24. T. J. Allen, B. T. Cox, and P. C. Beard, “Generating photoacoustic signals using high-peak power pulsed laser diodes,” in Proceedings of SPIE (2005), pp. 233-242.
25. R. G. M. Kolkman, W. Steenbergen, and T. G. van Leeuwen, “In vivo photoacoustic imaging of blood vessels with a pulsed laser diode,” Lasers Med. Sci. 21, 134-139 (2006).
26. L. Zeng, G. Liu, D. Yang, and X. Ji, “3D-visual laser-diode-based photoacoustic imaging,” Opt. Express 20, 1237-1246 (2012).
27. T. Wang, S. Nandy, H. S. Salehi, P. D. Kumavor, and Q. Zhu, “A low-cost photoacoustic microscopy system with a laser diode excitation,” Biomed. Opt. Express 5, 3053-3058 (2014).
28. P. Leboulluec, H. Liu, and B. Yuan, “A cost-efficient frequency-domain photoacoustic imaging system,” Am. J. Phys. 81, 712 (2013).
29. K. Maslov, and L. V. Wang, “Photoacoustic imaging of biological tissue with intensity-modulated continuous-wave laser,” J. Biomed. Opt. 13, 024006 (2008).
30. K. Daoudi, P. J. van den Berg, O. Rabot, A. Kohl, S. Tisserand, P. Brands, and W. Steenbergen, “Handheld probe integrating laser diode and ultrasound transducer array for ultrasound/photoacoustic dual modality imaging,” Opt. Express 22, 26365 (2014).
31. G. Ku, X. Wang, G. Stoica, and L. V. Wang, “Multiple-bandwidth photoacoustic tomography,” Physics in Medicine and Biology 49, 1329-1338 (2004).
32. R. A. Kruger, P. Liu, Y. R. Fang, and C. R. Appledorn, “Photoacoustic ultrasound (PAUS)—reconstruction tomography,” Med. Phys. 22, 1605-1609 (1995).
33. R. Ma, A. Taruttis, V. Ntziachristos, and D. Razansky, “Multispectral optoacoustic tomography (MSOT) scanner for whole-body small animal imaging,” Opt. Express 17, 21414-21426 (2009).
34. American National Standard for Safe Use of Lasers, ANSI Standard Z136.1-2000, NY (2000).
35. C. Kim, C. Favazza, and L. V. Wang, “In vivo photoacoustic tomography of chemicals: high-resolution functional and molecular optical imaging at new depths,” Chem. Rev. 110, 2756-2782 (2010).
36. M. Pramanik, M. Swierczewska, D. Green, B. Sitharaman, and L. V. Wang, “Single-walled carbon nanotubes as a multimodal-thermoacoustic and photoacoustic-contrast agent,” J. Biomed. Opt. 14, 034018 (2009).
37. D. Pan, M. Pramanik, A. Senpan, X. Yang, K. H. Song, M. J. Scott, H. Zhang, P. J. Gaffney, S. A. Wickline, L. V. Wang, and G. M. Lanza, “Molecular photoacoustic tomography with colloidal nanobeacons,” Angew. Chem. Int. Ed. Engl. 48, 4170-4173 (2009).
38. Ermilov S A, Khamapirad T, Conjusteau A, Leonard M H, Lacewell R, Mehta K, et al. Laser optoacoustic imaging system for detection of breast cancer. J Biomed Opt. 2009; 14(2):024007.
39. Cai X, Kim C, Pramanik M, Wang L V. Photoacoustic tomography of foreign bodies in soft biological tissue. J Biomed Opt. 2011; 16(4):046017.
40. Su Y X, Zhang F, Xu K X, Yao J Q, Wang R K K. A photoacoustic tomography system for imaging of biological tissues. J Phys D Appl Phys. 2005; 38(15):2640-4.
41. Wang L, Xia J, Yao J, Maslov K I, Wang L V. Ultrasonically Encoded Photoacoustic Flowgraphy in Biological Tissue. Physical Review Letters. 2013; 111(20).
42. Xia J, Chatni M R, Maslov K, Guo Z, Wang K, Anastasio M, et al. Whole-body ring-shaped confocal photoacoustic computed tomography of small animals in vivo. J Biomed Opt. 2012; 17(5):050506.
43. Hu S, Wang L V. Photoacoustic imaging and characterization of the microvasculature. J Biomed Opt. 2010; 15(1):011101.
44. Upputuri P K, Wen Z-B, Wu Z, Pramanik M. Super-resolution photoacoustic microscopy using photonic nanojets: a simulation study. J Biomed Opt. 2014; 19(11):116003.
45. Danielli A, Maslov K, Garcia-Uribe A, Winkler A M, Li C, Wang L, et al. Label-free photoacoustic nanoscopy. J Biomed Opt. 2014; 19(8):086006.
46. Wang L, Zhang C, Wang L V. Grueneisen Relaxation Photoacoustic Microscopy. Phys Rev Lett. 2014; 113(17):174301.
47. Wang X, Pang Y, Ku G, Xie X, Stoica G, Wang L V. Non-invasive laser-induced photoacoustic tomography for structural and functional imaging.pdf. Nature Biotechnology. 2003; 21:803-6.
48. Kolkman R G M, Thumma K K, ten Brinke G A, Siphanto R I, van Neck H, Steenbergen W, et al. Photoacoustic imaging of tumor angiogenesis: Spie; 2008.
49. Pan D, Pramanik M, Senpan A, Allen J S, Zhang H, Wickline S A, et al. Molecular photoacoustic imaging of angiogenesis with integrin-targeted gold nanobeacons. FASEB J. 2011; 25(3):875-82.
50. Bosschaart N, Kolkman R, van Leeuwen T, Steenbergen W, editors. Imaging of venous valves with photoacoustics. Proceedings of SPIE; 2006.
51. Xie Z, Roberts W, Carson P, Liu X, Tao C, Wang X. Evaluation of bladder microvasculature with high-resolution photoacoustic imaging. Optics Letters. 2011; 36:4815-7.
52. Wang T, Yang Y, Alqasemi U, Kumavor P D, Wang X, Sanders M, et al. Characterization of ovarian tissue based on quantitative analysis of photoacoustic microscopy images. Biomed Opt Express. 2013; 4(12):2763-8.
53. Pan D, Pramanik M, Senpan A, Ghosh S, Wickline S A, Wang L V, et al. Near infrared photoacoustic detection of sentinel lymph nodes with gold nanobeacons. Biomaterials. 2010; 31(14):4088-93.
54. Pramanik M, Song K H, Swierczewska M, Green D, Sitharaman B, Wang L V. In vivo carbon nanotube-enhanced non-invasive photoacoustic mapping of the sentinel lymph node. Phys Med Biol. 2009; 54(11):3291-301.
55. Grootendorst D J, Jose J, Wouters M W, van Boven H, Van der Hage J, Van Leeuwen T G, et al. First experiences of photoacoustic imaging for detection of melanoma metastases in resected human lymph nodes. Lasers Surg Med. 2012; 44(7):541-9.
56. Pramanik M, Wang L V. Thermoacoustic and photoacoustic sensing of temperature. J Biomed Opt. 2009; 14(5):054024.
57. Cai X, Zhang Y S, Xia Y, Wang L V. Photoacoustic Microscopy in Tissue Engineering. Materials today. 2013; 16(3):67-77.
58. Talukdar Y, Avti P, Sun J, Sitharaman B. Multimodal ultrasound-photoacoustic imaging of tissue engineering scaffolds and blood oxygen saturation in and around the scaffolds. Tissue engineering Part C, Methods. 2014; 20(5):440-9
59. Ke H, Erpelding T N, Jankovic L, Liu C, Wang L V. Performance characterization of an integrated ultrasound, photoacoustic, and thermoacoustic imaging system. J Biomed Opt. 2012; 17(5):056010.
60. Robb R A. 3-D visualization in biomedical applications. Annual Review of Biomedical Engineering. 1999; 1(1):377-99.
61. Zhang H F, Maslov K, Wang L H V. In vivo imaging of subcutaneous structures using functional photoacoustic microscopy. Nat Protoc. 2007; 2(4):797-804.
62. Wang L V, Wu H-I. Biomedical Optics: Principles and Imaging. New Jersey: John Wiley & Sons, Inc.; 2009.
63. Ku G, Wang L V. Deeply penetrating photoacoustic tomography in biological tissues enhanced with an optical contrast agent. Opt Lett. 2005; 30(5):507-9.
64. Pan D, Pramanik M, Wickline S A, Wang L V, Lanza G M. Recent advances in colloidal gold nanobeacons for molecular photoacoustic imaging. Contrast Media Mol Imaging. 2011; 6(5):378-88.
65. Pan D, Pramanik M, Senpan A, Wickline S A, Wang L V, Lanza G M. A facile synthesis of novel self-assembled gold nanorods designed for near-infrared imaging. Journal of Nanoscience and Nanotechnology. 2010; 10(12):8118-23.
66. Kim C, Qin R, Xu J S, Wang L V, Xu R. Multifunctional microbubbles and nanobubbles for photoacoustic and ultrasound imaging. J Biomed Opt. 2010; 15(1):010510.
67. Hebden, J. C., Arridge, S. R., and Delpy, D. T., “Optical imaging in medicine 0.1. Experimental techniques,” Physics in Medicine and Biology, 42(5), 825-840 (1997).
68. Boas, D. A., Brooks, D. H., Miller, E. L. et al., “Imaging the body with diffuse optical tomography,” IEEE Signal Processing Magazine, 18(6), 57-75 (2001).
69. Dean, J., Gornstein, V., Burcher, M. et al., “Real-time photoacoustic data acquisition with Philips iU22 ultrasound scanner.” 6856, 685622.
70. Wang, L. V., Zhao, X., Sun, H. et al., “Microwave-induced acoustic imaging of biological tissues,” Review of Scientific Instruments, 70(9), 3744-48 (1999).
71. Lalwani, G., Cai, X., Nie, L. et al., “Graphene-based contrast agents for photoacoustic and thermoacoustic tomography,” Photoacoustics, 1(3-4), 62-67 (2013).
72. Xu, M., and Wang, L. V., “Time-domain reconstruction for thermoacoustic tomography in a spherical geometry,” IEEE Transactions on Medical Imaging, 21(7), 814-22 (2002).
73. Xu, Y., Feng, D. Z., and Wang, L. V., “Exact frequency-domain reconstruction for thermoacoustic tomography—I: Planar geometry,” IEEE Transactions on Medical Imaging, 21(7), 823-828 (2002).
74. Xu, Y., Xu, M. H., and Wang, L. V., “Exact frequency-domain reconstruction for thermoacoustic tomography—II: Cylindrical geometry,” IEEE Transactions on Medical Imaging, 21(7), 829-833 (2002).
75. Shaw, C. B., Prakash, J., Pramanik, M. et al., “Least squares QR-based decomposition provides an efficient way of computing optimal regularization parameter in photoacoustic tomography,” Journal of Biomedical Optics, 18(8), 080501 (2013).
76. Pramanik, M., “Improving tangential resolution with a modified delay-and-sum reconstruction algorithm in photoacoustic and thermoacoustic tomography,” Journal of the Optical Society of America A, 31(3), 621-7 (2014).
77. Aguirre, A., Gamelin, J., Guo, P. et al., “Feasibility study of three-dimensional co-registered ultrasound and photoacoustic imaging for cancer detection and visualization,” Proceedings of SPIE 6856, 68562A (2008).
78. Ashkenazi, S., Huang, S.-W., Horvath, T. et al., “Oxygen sensing for in vivo imaging by photoacoustic lifetime probing,” Proceedings of SPIE 6856, 68560D (2008).
79. Daoudi, K., van den Berg, P. J., Rabot, O. et al., “Handheld probe for portable high frame photoacoustic/ultrasound imaging system,” Proceedings of SPIE 8581, 858121 (2013).
80. Rabasović, M. D., Nikolić, M. G., Dramićanin, M. D. et al., “Low-cost, portable photoacoustic setup for solid samples,” Measurement Science and Technology, 20(9), 095902 (2009).
81. Wang, X., Xie, X., Ku, G. et al., “Noninvasive imaging of hemoglobin concentration and oxygenation in the rat brain using high-resolution photoacoustic tomography,” Journal of Biomedical Optics, 11(2), 024015 (2006).
82. Xia, J., Wang, Y., and Wan, H., “Recent Progress in Multimodal Photoacoustic Tomography,” X-Acoustics: Imaging and Sensing, 1(1), (2015).
83. Xia1, J., Kim, C., and Lovell, J. F., “Opportunities for Photoacoustic-Guided Drug Delivery,” Current Drug Targets, 16, 571-581 (2015).
84. Kim, J., Lee, D., Jung, U. et al., “Photoacoustic imaging platforms for multimodal imaging,” Ultrasonography, 34(2), 88-97 (2015).
85. Upputuri, P. K., Sivasubramanian, K., Mark, C. S. K. et al., “Recent Developments in Vascular Imaging Techniques in Tissue Engineering and Regenerative Medicine,” BioMed Research International, 2015, 9 (2015).
86. Song, K. H., and Wang, L. V., “Deep reflection-mode photoacoustic imaging of biological tissue,” Journal of Biomedical Optics, 12(6), 060503 (2007).
87. Xia, J., and Wang, L. V., [Photoacoustic Tomography of the Brain] Springer Science+Business Media New York, 6 (2013).
88. Yang, X., and Wang, L. V., “Monkey brain cortex imaging by photoacoustic tomography,” Journal of Biomedical Optics, 13(4), 044009 (2008).
89. Hu, S., Maslov, K., Tsytsarev, V. et al., “Functional transcranial brain imaging by optical-resolution photoacoustic microscopy,” Journal of Biomedical Optics, 14(4), (2009).
90. Li, T., Xu, X., Chen, B. et al., “Photoacoustic imaging of acupuncture effect in small animals,” Biomedical Optics Express, 6(2), 433-442 (2015).
91. Song, K. H., Stoica, G., and Wang, L. V., “In vivo three-dimensional photoacoustic tomography of a whole mouse head,” Optics Letters, 31(16), 2453-5 (2006).
92. Stein, E. W., Maslov, K., and Wang, L. V., “Noninvasive, in vivo imaging of blood-oxygenation dynamics within the mouse brain using photoacoustic microscopy,” Journal of Biomedical Optics, 14(2), 020502 (2009).
93. Wang, X., Ku, G., Wegiel, M. A. et al., “Noninvasive photoacoustic angiography of animal brains in vivo with near-infrared light and an optical contrast agent,” Optics Letters, 29, 730-702 (2004).
94. Diot, G., Dima, A., and Ntziachristos, V., “Multispectral opto-acoustic tomography of exercised muscle oxygenation,” Optics Letters, 40(7), 1496-1499 (2015).
95. Razansky, D., Distel, M., Vinegoni, C. et al., “Multispectral opto-acoustic tomography of deep-seated fluorescent proteins in vivo,” Nature Photonics, 3(7), 412-417 (2009).
96. Upputuri, P. K., and Pramanik, M., “Performance characterization of low-cost, high-speed, portable pulsed laser diode photoacoustic tomography (PLD-PAT) system,” Biomedical Optics Express, 6(10), 4118-29 (2015).
97. Upputuri, P. K., and Pramanik, M., “Pulsed laser diode based optoacoustic imaging of biological tissues,” Biomedical Physics & Engineering Express, 1(4), 045010 (2015).
98. Upputuri, P. K., Sivasubramanian, K., and Pramanik, M., “High speed photoacoustic tomography system with low cost portable pulsed diode laser,” Proceedings of SPIE, 9524, 95240G-1-95240G-7 (2015).
99. Zhisong Wang, J. Li, and R. Wu, “Time-Delay- and Time-Reversal-Based Robust capon beam formers for ultrasound Imaging,” IEEE Transactions on medical imaging 24(10), 1308-1322 (2005).
100. High power visible light emitting diodes as pulsed excitation sources for biomedical photoacoustics,” Biomedical Optics Express, 7(4), 1260-1270 (2016).

1. A photo-acoustic imaging apparatus for imaging a region of a subject, the photo-acoustic apparatus comprising:
a light source for directing light at the region of the subject; and
a photo-acoustic transducer for sensing photo-acoustic signals induced in the region of the subject by the light, the photo-acoustic transducer being immersed in an ultrasound coupling medium and arranged to scan the region of the subject and to move in a curvilinear path around the region of the subject; wherein
the light source is disposed within a volume defined, at least in part, by the curvilinear path.

2. The photo-acoustic imaging apparatus of claim 1 arranged for the photo-acoustic transducer to sense induced photo-acoustic signals at plural points along the curvilinear path and, optionally, to perform signal averaging of the sensed induced photo-acoustic signals.

3. The photo-acoustic imaging apparatus of claim 1 arranged for a sensor of the photo-acoustic transducer to face the region of the subject at plural points along the curvilinear path.

4. The photo-acoustic imaging apparatus of claim 1 arranged for a distance between the light source and the region of the subject to be selectively variable.

5. The photo-acoustic imaging apparatus of claim 1, wherein the curvilinear path has a radius and the apparatus is configured for the radius to be selectively variable.

6. The photo-acoustic imaging apparatus of claim 1, wherein the light source comprises a high-repetition rate pulsed laser diode.

7. The photo-acoustic imaging apparatus of claim 1, wherein the apparatus comprises a plural number of photo-acoustic transducers, wherein a distance of travel of each of the plural photo-acoustic transducers along the curvilinear path is defined as a full distance of the curvilinear path modified by the plural number of photo-acoustic transducers.

8. The photo-acoustic imaging apparatus of claim 1, further comprising a guide member for guiding induced photo-acoustic signals to the photo-acoustic transducer.

9. The photo-acoustic imaging apparatus of claim 1, wherein the ultrasound coupling medium comprises water.

10. A method of imaging a region of a subject, the method comprising:
directing light from a light source at the region of the subject;
sensing photo-acoustic signals induced in the region of the subject by the light using a photo-acoustic transducer immersed in an ultrasound coupling medium, the photo-acoustic transducer scanning the region of the subject and moving in a curvilinear path around the region of the subject, wherein
the light source is disposed within a volume defined, at least in part, by the curvilinear path.
